(19) United States
(12) Patent Application Publication
(10) Pub. No.: US 2026/0218228 A1
(43) Pub. Date: Jul. 30, 2026

(54) RECOMBINANT ADENO-ASSOCIATED VIRUS VECTOR

(71) Applicant: QUETHERA LIMITED, Addlestone Kent (GB)

(72) Inventors: Kenjiro ADACHI, Tokyo (JP); Naoki KOZONO, Tokyo (JP)

(21) Appl. No.: 19/142,377

(22) PCT Filed: Feb. 1, 2024
(86) PCT No.: PCT/GB2024/050275

§ 371 Date: Jun. 23, 2025

Related U.S. Application Data
(60) Provisional application No. 63/483,002, filed on Feb. 2, 2023.
(60) Provisional application No. 63/583,776, filed on Sep. 19, 2023.

(51) Int. Cl. C12N 15/86; A61K 38/17; A61K 38/18; A61K 48/00; A61P 27/06; C07K 14/475; C07K 14/71

(52) U.S. Cl. CPC C12N 15/86; A61K 38/179; A61K 38/185; A61K 48/005; A61P 27/06; C07K 14/475; C07K 14/71; C12N 2750/14143; C12N 2750/14151; C12N 2830/48; C12N 2830/50

(57) ABSTRACT

Some embodiments relate to adeno-associated virus (rAAV) vectors, in particular rAAV vectors comprising a genetic construct harbouring genes encoding tyrosine receptor kinase B and Brain Derived Neurotrophic Factor. The presently disclosed subject matter also extends to a pharmaceutical composition comprising the rAAV vectors, and to the use of such vectors and compositions in gene therapy methods for preventing or treating a range of optic nerve disorders and cochlear disorders, or for promoting nerve regeneration and/or survival. The presently disclosed subject matter also provides methods of producing the rAAV vectors.

### CROSS-REFERENCE TO RELATED APPLICATIONS

This application is a national phase filing under 35 C.F.R. § 371 of and claims priority to PCT Patent Application No. PCT/GB2024/050275, filed on Feb. 1, 2024, which claims the benefit of U.S. Provisional Application No. 63/483,002, filed on Feb. 2, 2023, and U.S. Provisional Application No. 63/583,776, filed on Sep. 19, 2023, the disclosure of each is hereby incorporated by reference in their entirety.

The presently disclosed subject matter relates to recombinant adeno-associated virus (rAAV) vectors, in particular rAAV vectors comprising a genetic construct harbouring genes encoding tyrosine receptor kinase B (TrkB) and Brain Derived Neurotrophic Factor (BDNF). The presently disclosed subject matter also extends to a pharmaceutical composition comprising the rAAV vector, and to the use of such vectors and compositions in gene therapy methods for preventing or treating a range of optic nerve disorders and cochlear disorders, or for promoting nerve regeneration and/or survival. The presently disclosed subject matter also provides methods of producing the rAAV vectors.

Retinal ganglion cells (RGCs) are cells that serve as the final pathway for transmitting all visual information processed by the retina to the brain. RGCs are cells primarily affected in optic neuropathy or optic neuritis including glaucoma (also referred to as glaucomatous optic neuropathy), hereditary optic nerve disorder, ischemic optic nerve disorder, and neurodegenerative disease (Int. J. Mol. Sci., 2020. 21(7): 2262; Hum. Mol. Genet., 2017. 26(R2): p. R139-R150). RGCs have limited regenerative ability, and hence blindness following optic nerve disorder is known to be irreversible (Science. 2017. 356(6342): p. 1031-1034).

Glaucomatous optic neuropathy, the most common optic nerve disorder, is a progressive optic nerve degeneration characterised by axonal damage of RGCs and accompanying death of RGCs, and causes loss of vision (Nat. Rev. Dis. Primers, 2016. 2: p. 16067; JAMA, 2014. 311(18): p. 1901-1911). Glaucoma, which includes open angle glaucoma, normal tension glaucoma, angle-closure glaucoma, congenital glaucoma, and secondary glaucoma, is a primary cause for irreversible loss of vision in the world. The incidence rate of glaucoma increases with age, and the global prevalence of glaucoma in 2013 for the population aged between 40 and 80 years was estimated to be approximately 3.5%, approximately 64.3 million cases, and predicted to increase to approximately 76.0 million cases by 2020, and to 111.8 million cases by 2040 (Ophthalmology, 2014. 121(11): p. 2081-2090). Currently, the elderly population is rapidly increasing, and therefore, glaucoma is an urgent social and medical problem.

The elevation of intraocular pressure (IOP) is the most important risk factor for glaucoma (Surv. Ophthalmol., 2003. 48 (Supplement 1): p. S3-S7). Current glaucoma treatments are based on the prevention of additional optic nerve injury by lowering IOP with topically applied drugs (Lancet, 1999. 354(9192): p. 1803-1810). Major agents primarily used to lower IOP are the following five types: β-adrenergic receptor antagonists, adrenergic receptor agonists, parasympathomimetic agents, prostaglandin analogs, and carbonic anhydrase inhibitors. In spite of the effect to lower IOP, these agents may cause severe side effects in some patients, adversely affecting their quality of life. In addition, compliance and adherence for administration of an eye drop to lower IOP is not high, particularly in patients who need to use multiple agents. When the degree of IOP lowering is insufficient and it is needed to further lower IOP, laser trabeculoplasty is occasionally carried out, however, even this method cannot lower IOP in many patients. Accordingly, protection of RGCs and their axons in glaucoma is an important therapeutic method to be used in addition to conventional IOP-lowering treatments, and particularly important for patients who have not benefited from conventional therapeutic methods (Eye (Lond), 2018. 32(5): p. 938-945).

Brain-derived neurotrophic factor (BDNF) is a member of the neurotrophin family of growth factors, along with nerve growth factor (NGF), neurotrophin-3 (NT-3), and neurotrophin-4/5 (NT-4/5) (Neuropathol. Appl. Neurobiol., 2003. 29(3): p. 211-230; Nat. Rev. Neurosci., 2003. 4(4): p. 299-309). Neurotrophins play an important role in development, survival, and function of a wide variety of neurons in the peripheral and central nervous systems. Neurotrophins bind to the two families of cell-surface receptors, the p75 neurotrophin receptor (p75^{NTR}) and the tropomyosin-related kinase (Trk) receptors. NGF mainly binds to TrkA and BDNF, NT-4/5 binds to TrkB, and NT-3 mainly binds to TrkC.

BDNF is one of the neurotrophins that can prevent the death of RGCs after axonal damage in the most effective manner (Invest. Ophthalmol. Vis. Sci., 1996. 37(4): p. 489-500; Invest. Ophthalmol. Vis. Sci., 2001. 42(5): p. 966-974; Neurosci. Lett., 2001. 305(2): p. 139-142; J. Neurosci., 2000. 20(18): p. 6962-6967). BDNF is normally synthesised as pre-proBDNF that contains a signal peptide sequence (Nat. Rev. Neurosci., 2013. 14(1): p. 7-23). Thereafter, the signal peptide is cleaved and removed to convert pre-proBDNF into proBDNF. The N-terminal sequence of proBDNF is cleaved intra- or extracellularly, and as a result mature BDNF (mBDNF) is generated. It is known that while mBDNF activates the TrkB receptor to maintain cell survival, proBDNF preferentially activates the p75^{NTR} receptor to induce cell death (Nat. Rev. Neurosci., 2005. 6(8): p. 603-614).

Animal models of glaucoma have demonstrated reduction of BDNF in the retina after optic nerve crush or an increase in IOP (Int. J. Mol. Sci., 2020. 21(17): 6262; Invest. Ophthalmol. Vis. Sci., 2000. 41(3): p. 764-774; Invest. Ophthalmol. Vis. Sci., 2000. 41(11): p. 3460-3466). In animal models of glaucoma, supplementation of intraocular BDNF by administration of a recombinant protein or by gene therapy, can increase the survival rate of RGCs as compared with untreated cases (Invest. Ophthalmol. Vis. Sci., 2001. 42(5): p. 966-974; Neurosci. Lett., 2001. 305(2): p. 139-142; J. Neurosci., 2000. 20(18): p. 6962-6967; Int. J. Mol. Sci., 2020. 21(17): 6262). It has been suggested, on the other hand, that the protective action for RGCs by supplementation of BDNF alone is expected to be exhibited only as a transient effect because of the down-regulation of the TrkB receptor (Int. J. Mol. Sci. 2019. 20(17): 4314). In such circumstances, for the purpose of successfully sustaining the effect of BDNF in the retina for a long period of time, rAAV vectors have been made in which a CAG promoter drives expression of a TrkB gene and a BDNF gene.

However, the inventors of the presently disclosed subject matter have observed an important discrepancy in the yield when manufacturing the prior art rAAV vectors designed in accordance with the teaching of International Publication No. WO 2017/072498 and Hum. Gene Ther., 2018. 29(7): p. 828-841. In particular, the inventors observed a problem in which rAAV vectors comprising a TrkB gene and a BDNF gene, as designed in accordance with the teaching of the documents, showed genome fragmentation (or truncation) of rAAV genomic DNA in the production process. The occurrence of the fragmentation of genomic DNA significantly interferes with efficient production of a rAAV vector comprising a TrkB gene and a BDNF gene, resulting in lowered production efficiency of the rAAV vector, and reduced yields.

Therefore, the inventors set out to design and produce rAAV vectors comprising both a TrkB gene and a BDNF gene, but which does not experience the problem of truncation or fragmentation of the genomic DNA. The inventors observed that rAAV vectors carrying a cytomegalovirus (CMV) promoter operably linked to a TrkB gene and a mature BDNF gene, demonstrated reduced fragmentation of genomic DNA, and so a higher efficiency of the rAAV vectors was observed, resulting in better yields.

Thus, according to a first aspect of the presently disclosed subject matter, there is provided a recombinant adeno-associated virus (rAAV) vector comprising a genetic construct comprising, in a 5′ to 3′ orientation:
a cytomegalovirus (CMV) promoter;
a first coding sequence, which encodes tyrosine kinase receptor B (TrkB);
a nucleotide sequence encoding a linker to generate TrkB and mature brain-derived neurotrophic factor (mBDNF) as individual proteins; and
a second coding sequence, which encodes mBDNF,
   wherein the CMV promoter is operably linked to the first and second coding sequences.

Advantageously, the rAAV vector carrying a TrkB gene and a mature BDNF gene, and a CMV promoter operably linked to these genes, can reduce the truncation or fragmentation of genomic DNA in the production process. As such, the rAAV vector of the claimed presently disclosed subject matter can be produced with increased production efficiency. Pharmaceutical compositions comprising the rAAV vector can be used for prevention or treatment of optic nerve disorders and/or retinal degenerative diseases involving retinal ganglion cell degeneration, such as glaucoma and glaucomatous optic neuropathy.

The CMV promoter is operably linked to the first coding sequence, which encodes the tyrosine kinase receptor B (TrkB), and the second coding sequence, which encodes mature brain-derived neurotrophic factor (mBDNF). Herein, “operably linked” means that a promoter sequence is linked to the first and second coding sequence in such a manner that a protein encoded by the coding sequences can be expressed in host cells.

In one embodiment, the CMV promoter comprises a nucleotide sequence including a TATA box sequence derived from the CMV IE promoter and a CMV-derived sequence.

One embodiment of the nucleotide sequence encoding the CMV promoter is referred to herein as SEQ ID No: 1, as follows:

[SEQ ID No: 1]
ttaatagtaa tcaattacgg ggtcattagt tcatagccca tatatggagt tccgcgttac
ataacttacg gtaaatggcc cgcctggctg accgcccaac gacccccgcc cattgacgtc
aataatgacg tatgttccca tagtaacgcc aatagggact ttccattgac gtcaatgggt
ggagtattta cggtaaactg cccacttggc agtacatcaa gtgtatcata tgccaagtac
gccccctatt gacgtcaatg acggtaaatg gcccgcctgg cattatgccc agtacatgac
cttatgggac tttcctactt ggcagtacat ctacgtatta gtcatcgcta ttaccatggt
gatgcggttt tggcagtaca tcaatgggcg tggatagcgg tttgactcac ggggatttcc
aagtctccac cccattgacg tcaatgggag tttgttttgg caccaaaatc aacgggactt
tccaaaatgt cgtaacaact ccgccccatt gacgcaaatg ggcggtaggc gtgtacggtg
ggaggtctat ataagcagag ctggtttagt g

In one embodiment, therefore, the CMV promoter comprises a nucleotide sequence as set out in SEQ ID No: 1, or a fragment or variant thereof.

The genetic construct comprised in the rAAV vector of the presently disclosed subject matter, in one embodiment, comprises a first coding sequence encoding naturally occurring TrkB, or a variant having the function thereof. It will be well understood by the skilled person that “naturally occurring” TrkB, describes the gene when found in its natural form, without the introduction of any unnatural mutations or modifications.

TrkB has a function to activate intracellular signalling molecules (e.g., extracellular signal-regulated kinase (ERK)) downstream of TrkB upon binding to BDNF and neurotrophin-4/5 (NT-4/5). The function of TrkB can be evaluated by using a method known to those skilled in the art such as a ligand binding assay and detection of the activity of an intracellular signalling molecule. The nucleotide sequence encoding TrkB is, in some embodiments, a nucleotide sequence encoding mammalian TrkB, and, in some embodiments, a nucleotide sequence encoding human TrkB.

In one embodiment, TrkB comprises an amino acid sequence referred to herein as SEQ ID No: 2 (accession No. NP_001018074.1), as follows:

[SEQ ID NO: 2]
Met Ser Ser Trp Ile Arg Trp His Gly Pro Ala Met Ala Arg Leu Trp
Gly Phe Cys Trp Leu Val Val Gly Phe Trp Arg Ala Ala Phe Ala Cys
Pro Thr Ser Cys Lys Cys Ser Ala Ser Arg Ile Trp Cys Ser Asp Pro
Ser Pro Gly Ile Val Ala Phe Pro Arg Leu Glu Pro Asn Ser Val Asp
Pro Glu Asn Ile Thr Glu Ile Phe Ile Ala Asn Gln Lys Arg Leu Glu
Ile Ile Asn Glu Asp Asp Val Glu Ala Tyr Val Gly Leu Arg Asn Leu
Thr Ile Val Asp Ser Gly Leu Lys Phe Val Ala His Lys Ala Phe Leu
Lys Asn Ser Asn Leu Gln His Ile Asn Phe Thr Arg Asn Lys Leu Thr
Ser Leu Ser Arg Lys His Phe Arg His Leu Asp Leu Ser Glu Leu Ile
Leu Val Gly Asn Pro Phe Thr Cys Ser Cys Asp Ile Met Trp Ile Lys
Thr Leu Gln Glu Ala Lys Ser Ser Pro Asp Thr Gln Asp Leu Tyr Cys
Leu Asn Glu Ser Ser Lys Asn Ile Pro Leu Ala Asn Leu Gln Ile Pro
Asn Cys Gly Leu Pro Ser Ala Asn Leu Ala Ala Pro Asn Leu Thr Val
Glu Glu Gly Lys Ser Ile Thr Leu Ser Cys Ser Val Ala Gly Asp Pro
Val Pro Asn Met Tyr Trp Asp Val Gly Asn Leu Val Ser Lys His Met
Asn Glu Thr Ser His Thr Gln Gly Ser Leu Arg Ile Thr Asn Ile Ser
Ser Asp Asp Ser Gly Lys Gln Ile Ser Cys Val Ala Glu Asn Leu Val
Gly Glu Asp Gln Asp Ser Val Asn Leu Thr Val His Phe Ala Pro Thr
Ile Thr Phe Leu Glu Ser Pro Thr Ser Asp His His Trp Cys Ile Pro
Phe Thr Val Lys Gly Asn Pro Lys Pro Ala Leu Gln Trp Phe Tyr Asn
Gly Ala Ile Leu Asn Glu Ser Lys Tyr Ile Cys Thr Lys Ile His Val
Thr Asn His Thr Glu Tyr His Gly Cys Leu Gln Leu Asp Asn Pro Thr
His Met Asn Asn Gly Asp Tyr Thr Leu Ile Ala Lys Asn Glu Tyr Gly
Lys Asp Glu Lys Gln Ile Ser Ala His Phe Met Gly Trp Pro Gly Ile
Asp Asp Gly Ala Asn Pro Asn Tyr Pro Asp Val Ile Tyr Glu Asp Tyr
Gly Thr Ala Ala Asn Asp Ile Gly Asp Thr Thr Asn Arg Ser Asn Glu
Ile Pro Ser Thr Asp Val Thr Asp Lys Thr Gly Arg Glu His Leu Ser
Val Tyr Ala Val Val Val Ile Ala Ser Val Val Gly Phe Cys Leu Leu
Val Met Leu Phe Leu Leu Lys Leu Ala Arg His Ser Lys Phe Gly Met
Lys Gly Pro Ala Ser Val Ile Ser Asn Asp Asp Asp Ser Ala Ser Pro
Leu His His Ile Ser Asn Gly Ser Asn Thr Pro Ser Ser Ser Glu Gly
Gly Pro Asp Ala Val Ile Ile Gly Met Thr Lys Ile Pro Val Ile Glu
Asn Pro Gln Tyr Phe Gly Ile Thr Asn Ser Gln Leu Lys Pro Asp Thr
Phe Val Gln His Ile Lys Arg His Asn Ile Val Leu Lys Arg Glu Leu
Gly Glu Gly Ala Phe Gly Lys Val Phe Leu Ala Glu Cys Tyr Asn Leu
Cys Pro Glu Gln Asp Lys Ile Leu Val Ala Val Lys Thr Leu Lys Asp
Ala Ser Asp Asn Ala Arg Lys Asp Phe His Arg Glu Ala Glu Leu Leu
Thr Asn Leu Gln His Glu His Ile Val Lys Phe Tyr Gly Val Cys Val
Glu Gly Asp Pro Leu Ile Met Val Phe Glu Tyr Met Lys His Gly Asp
Leu Asn Lys Phe Leu Arg Ala His Gly Pro Asp Ala Val Leu Met Ala
Glu Gly Asn Pro Pro Thr Glu Leu Thr Gln Ser Gln Met Leu His Ile
Ala Gln Gln Ile Ala Ala Gly Met Val Tyr Leu Ala Ser Gln His Phe
Val His Arg Asp Leu Ala Thr Arg Asn Cys Leu Val Gly Glu Asn Leu
Leu Val Lys Ile Gly Asp Phe Gly Met Ser Arg Asp Val Tyr Ser Thr
Asp Tyr Tyr Arg Val Gly Gly His Thr Met Leu Pro Ile Arg Trp Met
Pro Pro Glu Ser Ile Met Tyr Arg Lys Phe Thr Thr Glu Ser Asp Val
Trp Ser Leu Gly Val Val Leu Trp Glu Ile Phe Thr Tyr Gly Lys Gln
Pro Trp Tyr Gln Leu Ser Asn Asn Glu Val Ile Glu Cys Ile Thr Gln
Gly Arg Val Leu Gln Arg Pro Arg Thr Cys Pro Gln Glu Val Tyr Glu
Leu Met Leu Gly Cys Trp Gln Arg Glu Pro His Met Arg Lys Asn Ile
Lys Gly Ile His Thr Leu Leu Gln Asn Leu Ala Lys Ala Ser Pro Val
Tyr Leu Asp Ile Leu Gly

In one embodiment, therefore, the first coding sequence encodes an amino acid sequence as set out in SEQ ID No: 2, or a fragment or variant thereof.

One embodiment of the nucleotide sequence encoding TrkB is referred to herein as SEQ ID No: 3, as follows:

[SEQ ID No: 3]
atgtcgtcct ggataaggtg gcatggaccc gccatggcgc ggctctgggg cttctgctgg
ctggttgtgg gcttctggag ggccgctttc gcctgtccca cgtcctgcaa atgcagtgcc
tctcggatct ggtgcagcga cccttctcct ggcatcgtgg catttccgag attggagcct
aacagtgtag atcctgagaa catcaccgaa attttcatcg caaaccagaa aaggttagaa
atcatcaacg aagatgatgt tgaagcttat gtgggactga gaaatctgac aattgtggat
tctggattaa aatttgtggc tcataaagca tttctgaaaa acagcaacct gcagcacatc
aattttaccc gaaacaaact gacgagtttg tctaggaaac atttccgtca ccttgacttg
tctgaactga tcctggtggg caatccattt acatgctcct gtgacattat gtggatcaag
actctccaag aggctaaatc cagtccagac actcaggatt tgtactgcct gaatgaaagc
agcaagaata ttcccctggc aaacctgcag atacccaatt gtggtttgcc atctgcaaat
ctggccgcac ctaacctcac tgtggaggaa ggaaagtcta tcacattatc ctgtagtgtg
gcaggtgatc cggttcctaa tatgtattgg gatgttggta acctggtttc caaacatatg
aatgaaacaa gccacacaca gggctcctta aggataacta acatttcatc cgatgacagt
gggaagcaga tctcttgtgt ggcggaaaat cttgtaggag aagatcaaga ttctgtcaac
ctcactgtgc attttgcacc aactatcaca tttctcgaat ctccaacctc agaccaccac
tggtgcattc cattcactgt gaaaggcaac cccaaaccag cgcttcagtg gttctataac
ggggcaatat tgaatgagtc caaatacatc tgtactaaaa tacatgttac caatcacacg
gagtaccacg gctgcctcca gctggataat cccactcaca tgaacaatgg ggactacact
ctaatagcca agaatgagta tgggaaggat gagaaacaga tttctgctca cttcatgggc
tggcctggaa ttgacgatgg tgcaaaccca aattatcctg atgtaattta tgaagattat
ggaactgcag cgaatgacat cggggacacc acgaacagaa gtaatgaaat cccttccaca
gacgtcactg ataaaaccgg tcgggaacat ctctcggtct atgctgtggt ggtgattgcg
tctgtggtgg gattttgcct tttggtaatg ctgtttctgc ttaagttggc aagacactcc
aagtttggca tgaaaggccc agcctccgtt atcagcaatg atgatgactc tgccagccca
ctccatcaca tctccaatgg gagtaacact ccatcttctt cggaaggtgg cccagatgct
gtcattattg gaatgaccaa gatccctgtc attgaaaatc cccagtactt tggcatcacc
aacagtcagc tcaagccaga cacatttgtt cagcacatca agcgacataa cattgttctg
aaaagggagc taggcgaagg agcctttgga aaagtgttcc tagctgaatg ctataacctc
tgtcctgagc aggacaagat cttggtggca gtgaagaccc tgaaggatgc cagtgacaat
gcacgcaagg acttccaccg tgaggccgag ctcctgacca acctccagca tgagcacatc
gtcaagttct atggcgtctg cgtggagggc gaccccctca tcatggtctt tgagtacatg
aagcatgggg acctcaacaa gttcctcagg gcacacggcc ctgatgccgt gctgatggct
gagggcaacc cgcccacgga actgacgcag tcgcagatgc tgcatatagc ccagcagatc
gccgcgggca tggtctacct ggcgtcccag cacttcgtgc accgcgattt ggccaccagg
aactgcctgg tcggggagaa cttgctggtg aaaatcgggg actttgggat gtcccgggac
gtgtacagca ctgactacta cagggtcggt ggccacacaa tgctgcccat tcgctggatg
cctccagaga gcatcatgta caggaaattc acgacggaaa gcgacgtctg gagcctgggg
gtcgtgttgt gggagatttt cacctatggc aaacagccct ggtaccagct gtcaaacaat
gaggtgatag agtgtatcac tcagggccga gtcctgcagc gaccccgcac gtgcccccag
gaggtgtatg agctgatgct ggggtgctgg cagcgagagc cccacatgag gaagaacatc
aagggcatcc ataccctcct tcagaacttg gccaaggcat ctccggtcta cctggacatt
ctaggc

In one embodiment, therefore, the first coding sequence comprises a nucleotide sequence as set out in SEQ ID No: 3, or a fragment or variant thereof.

The genetic construct comprised in the rAAV vector of the presently disclosed subject matter may comprise a second coding sequence encoding naturally occurring mature BDNF. It will be well understood by the skilled person that “naturally occurring” mBDNF, describes the gene when found its natural form, without the introduction of any unnatural mutations or modifications.

BDNF is a ligand for TrkB, and known to be present in the form of pre-proBDNF, proBDNF, or mature BDNF (mBDNF). Specifically, BDNF is first synthesized as pre-proBDNF as a precursor protein, which in turn is transferred into the rough endoplasmic reticulum and converted into proBDNF through cleavage of the signal peptide. proBDNF is converted into mBDNF through cleavage of the N-terminal peptide sequence. Both proBDNF and mBDNF are extracellularly secreted, of which proBDNF preferentially activates the p75^{NTR} receptor and mBDNF activates the TrkB receptor. The function of proBDNF or mBDNF can be evaluated using a method known to those skilled in the art such as a receptor binding assay and detection of the activity of an intracellular signalling molecule downstream of the receptor.

The nucleotide sequence encoding mBDNF is, in some embodiments, a nucleotide sequence encoding mammalian mBDNF, and in some embodiments, a nucleotide sequence encoding human mBDNF. Accordingly, in one embodiment, a nucleotide sequence encoding human mBDNF or a variant having the function thereof, can be used as a nucleotide sequence encoding human mBDNF.

In one embodiment, mBDNF comprises an amino acid sequence referred to herein as SEQ ID No: 4 (129 to 247 of amino acid sequence of accession No. NP_001137277.1), as follows:

[SEQ ID No: 4]
His Ser Asp Pro Ala Arg Arg Gly Glu Leu Ser Val Cys Asp Ser Ile
Ser Glu Trp Val Thr Ala Ala Asp Lys Lys Thr Ala Val Asp Met Ser
Gly Gly Thr Val Thr Val Leu Glu Lys Val Pro Val Ser Lys Gly Gln
Leu Lys Gln Tyr Phe Tyr Glu Thr Lys Cys Asn Pro Met Gly Tyr Thr
Lys Glu Gly Cys Arg Gly Ile Asp Lys Arg His Trp Asn Ser Gln Cys
Arg Thr Thr Gln Ser Tyr Val Arg Ala Leu Thr Met Asp Ser Lys Lys
Arg Ile Gly Trp Arg Phe Ile Arg Ile Asp Thr Ser Cys Val Cys Thr
Leu Thr Ile Lys Arg Gly Arg

In one embodiment, therefore, the second coding sequence encodes an amino acid sequence as set out in SEQ ID No: 4, or a fragment or variant thereof.

One embodiment of the nucleotide sequence encoding mBDNF is referred to herein as SEQ ID No: 5, as follows:

[SEQ ID No: 5]
cactctgacc ctgcccgccg aggggagctg agcgtgtgtg acagtattag tgagtgggta
acggcggcag acaaaaagac tgcagtggac atgtcgggcg ggacggtcac agtccttgaa
aaggtccctg tatcaaaagg ccaactgaag caatacttct acgagaccaa gtgcaatccc
atgggttaca caaaagaagg ctgcaggggc atagacaaaa ggcattggaa ctcccagtgc
cgaactaccc agtcgtacgt gcgggccctt accatggata gcaaaaagag aattggctgg
cgattcataa ggatagacac ttcttgtgta tgtacattga ccattaaaag gggaaga

In one embodiment, therefore, the second coding sequence comprises a nucleotide sequence as set out in SEQ ID No: 5, or a fragment or variant thereof.

As the rAAV vector of the presently disclosed subject matter includes a genetic construct encoding mBDNF, in some embodiments, the genetic construct further encodes a signal peptide. Accordingly, in some embodiments, the genetic construct comprised in the rAAV vector further comprises a nucleotide sequence encoding a signal peptide.

The nucleotide sequence encoding the signal peptide is positioned on the 5′ side of the nucleotide sequence encoding mBDNF. Accordingly, the genetic construct comprised in the rAAV vector of the presently disclosed subject matter includes, in a 5′ to 3′ direction, a nucleotide sequence encoding a signal peptide and the nucleotide sequence encoding mBDNF.

In one embodiment, the nucleotide sequence encoding the signal peptide is positioned on the 3′ side of the nucleotide sequence encoding the linker. Accordingly, in some embodiments, the genetic construct comprised in the rAAV vector of the presently disclosed subject matter includes, in a 5′ to 3′ direction, a cytomegalovirus (CMV) promoter, a nucleotide sequence encoding TrkB, a nucleotide sequence encoding a linker, a nucleotide sequence encoding a signal peptide, and a nucleotide sequence encoding mBDNF.

Any nucleotide sequence encoding a signal peptide with a function to promote extracellular secretion of mBDNF is applicable, without limitation, as the nucleotide sequence encoding a signal peptide for use in the presently disclosed subject matter, and examples thereof include nucleotide sequences encoding signal peptides described in WO 2017/072498, WO 2018/185468, Hum. Gene Ther., 2018. 29(7): p. 828-841, and Cell Death Dis., 2018. 9:1007. In one embodiment, the nucleotide sequence encoding a signal peptide is a nucleotide sequence encoding a natural amino acid sequence that is included at the N terminus of BDNF protein and has a function to promote extracellular secretion of proBDNF and mBDNF. In one embodiment, the nucleotide sequence encoding a signal peptide is a nucleotide sequence encoding an amino acid sequence that is obtained by modifying a natural amino acid sequence included at the N terminus of BDNF protein and has a function to promote extracellular secretion of proBDNF and mBDNF.

In one embodiment, the nucleotide sequence encoding a signal peptide is a nucleotide sequence encoding a natural amino acid sequence that is included at the N terminus of BDNF protein and has a function to promote extracellular secretion of proBDNF and mBDNF.

In some embodiments, the signal peptide comprises an amino acid sequence referred to herein as SEQ ID No: 20 (BDNF signal peptide: SP), as follows:

[SEQ ID No: 20]
Met Thr Ile Leu Phe Leu Thr Met Val Ile Ser Tyr Phe Gly Cys Met Lys
Ala

In one embodiment, therefore, the nucleotide sequence encoding the signal peptide encodes an amino acid sequence as set out in SEQ ID No: 20, or a fragment or variant thereof.

One embodiment of the nucleotide sequence encoding the signal peptide is referred to herein as SEQ ID No: 21, as follows:

[SEQ ID No: 21]
atgaccatcc ttttccttac tatggttatt tcatactttg gttgcatgaa ggct

In one embodiment, therefore, the signal peptide comprises a nucleotide sequence as set out in SEQ ID No: 21 or a fragment or variant thereof.

In one embodiment, the nucleotide sequence encoding a signal peptide is a nucleotide sequence encoding a signal peptide modified from a natural amino acid sequence that is included at the N terminus of BDNF protein and has a function to promote extracellular secretion of proBDNF and mBDNF.

In one embodiment, the signal peptide comprises an amino acid sequence referred to herein as SEQ ID No: 6 (nv3 signal peptide: mSP), as follows:

[SEQ ID No: 6]
Met Arg Ile Leu Leu Leu Thr Met Val Ile Ser Tyr Phe Gly Cys Met
Lys Ala

In one embodiment, therefore, the nucleotide sequence encoding the signal peptide encodes an amino acid sequence as set out in SEQ ID No: 6, or a fragment or variant thereof.

One embodiment of the nucleotide sequence encoding the signal peptide is referred to herein as SEQ ID No: 7, as follows:

[SEQ ID No: 7]
atgcggatcc ttctgcttac tatggttatt tcatactttg
gttgcatgaa ggct

In one embodiment, therefore, the signal peptide comprises a nucleotide sequence as set out in SEQ ID No: 7, or a fragment or variant thereof.

The genetic construct further comprises a nucleotide sequence encoding a linker to generate TrkB and mBDNF as individual proteins. The linker is disposed between the nucleotide sequence encoding TrkB and the nucleotide sequence encoding mBDNF. Accordingly, the genetic construct comprised in the rAAV vector of the presently disclosed subject matter includes, in a 5′ to 3′ direction, a nucleotide sequence encoding TrkB, a nucleotide sequence encoding a linker to generate TrkB and mBDNF as individual proteins, and a nucleotide sequence encoding mBDNF.

Herein, the “linker to generate TrkB and mBDNF as individual proteins” refers to a linker that allows a gene sequentially encoding two proteins to be translated to two individual proteins by ribosome skipping in host cells, or such a linker that after two proteins are translated as a single polypeptide, the two proteins can be then released as individual proteins through digestion or cleavage of the linker portion in host cells. In some embodiments, the linker can be digested or cleaved to thereby produce the Trkb and mBDNF as separate proteins.

The nucleotide sequence encoding the linker is a nucleotide sequence encoding a virus-derived peptide, specifically, a nucleotide sequence encoding a P2A peptide. The P2A peptide is a 2A peptide derived from porcine teschovirus-1.

In one embodiment, the nucleotide sequence encoding the linker may be a nucleotide sequence encoding a linker comprising a 2A peptide and an additional linker peptide. Accordingly, in one embodiment, the nucleotide sequence encoding a linker includes a nucleotide sequence encoding a linker comprising a 2A peptide and further an additional linker peptide. Any linker peptide that allows the linker to generate TrkB and BDNF as two individual proteins is applicable, without limitation, as the additional linker peptide, and examples thereof comprise a GSG (glycine-serine-glycine) sequence. Accordingly, in one embodiment, the nucleotide sequence encoding a linker is a nucleotide sequence encoding a linker consisting of a 2A peptide and GSG added to the N-terminus of the 2A peptide.

If the C-terminal amino acid of the polypeptide disposed at the N-terminus of the additional linker peptide is G, SG (serine-glycine) may be added, as an additional linker peptide, to the N-terminus of the 2A peptide. Accordingly, in one embodiment, the nucleotide sequence encoding a linker is a nucleotide sequence encoding a linker consisting of SG and a P2A peptide (herein, also referred to as an “SG-P2A peptide”).

In one embodiment, the linker comprises an amino acid sequence referred to herein as SEQ ID No: 8, as follows:

[SEQ ID No: 8]
Ser Gly Ala Thr Asn Phe Ser Leu Leu Lys Gln
Ala Gly Asp Val Glu Glu Asn Pro Gly Pro

In one embodiment, therefore, the nucleotide sequence encoding the linker encodes an amino acid sequence as set out in SEQ ID No: 8, or a fragment or variant thereof.

One embodiment of the nucleotide sequence encoding the linker is referred to herein as SEQ ID No: 9, as follows:

[SEQ ID No: 9]
agcggcgcca caaatttctc cctgctgaag caggcaggcg
acgtggagga gaaccctgga cca

In one embodiment, therefore, the linker comprises a nucleotide sequence as set out in SEQ ID No: 9, or a fragment or variant thereof.

In one embodiment, the genetic construct comprised in the rAAV vector of the presently disclosed subject matter further includes a post-transcriptional regulatory element. Herein, a “post-transcriptional regulatory element” refers to a non-coding sequence that regulates gene expression through post-transcriptional control. Any posttranscriptional regulatory element that is capable of regulating gene expression through post-transcriptional control is applicable, without limitation, as the post-transcriptional regulatory element that can be used in the presently disclosed subject matter, and examples thereof include a woodchuck hepatitis virus post-transcriptional regulatory element (WPRE). In one embodiment, therefore, the genetic construct comprises a nucleotide sequence encoding Woodchuck Hepatitis Virus Post-transcriptional Regulatory Element (WPRE), which enhances the expression of the two transgenes, i.e. the TrkB receptor and mBDNF. In one embodiment, the WPRE coding sequence is disposed 3′ of the transgene coding sequence, and in some embodiments, 3′ of the mBDNF coding sequence.

In some embodiments, the post-transcriptional regulatory element is a WPRE defined as a nucleotide sequence having a length of 247 bp (SEQ ID NO: 10) with the β element deleted (hereinafter, also referred to as WPRE(S)).

One embodiment of the nucleotide sequence encoding the WPRE is referred to herein as SEQ ID No: 10, as follows:

[SEQ ID No: 10]
aatcaacctc tggattacaa aatttgtgaa
agattgactg gtattcttaa ctatgttgct
ccttttacgc tatgtggata cgctgcttta
atgcctttgt atcatgctat tgcttcccgt
atggctttca ttttctcctc cttgtataaa
tcctggttag ttcttgccac ggcggaactc
atcgccgcct gccttgcccg ctgctggaca
ggggctcggc tgttgggcac tgacaattcc
gtggtgt

In one embodiment, therefore, the WPRE comprises a nucleotide sequence as set out in SEQ ID No: 10, or a fragment or variant thereof.

In one embodiment, the genetic construct comprised in the rAAV vector comprises a nucleotide sequence encoding a polyA signal sequence. Herein, “polyA signal sequences” are sequences that are known to those skilled in the art, and are DNA sequences that are disposed at the 3′ end of a gene and allow a polyadenosine (polyA) tail to be added to the 3′ end of mRNA transcribed from the gene. In one embodiment, the polyA signal sequence is a simian virus 40 (SV40) polyA signal sequence, a human β globin polyA signal sequence, a rabbit β globin polyA signal sequence, a bovine growth hormone polyA signal sequence, or a human growth hormone polyA signal sequence. In some embodiments, the polyA signal sequence is an SV40 polyA signal sequence.

In one embodiment, the polyA signal sequence is disposed 3′ of the transgene coding sequence, and in some embodiments, 3′ of the WPRE coding sequence.

One embodiment of the nucleotide sequence encoding the polyA signal sequence is referred to herein as SEQ ID No: 11, as follows:

[SEQ ID No: 11]
agacatgata agatacattg atgagtttgg
acaaaccaca actagaatgc agtgaaaaaa
atgctttatt tgtgaaattt gtgatgctat
tgctttattt gtaaccatta taagctgcaa
taaacaagtt aacaacaaca attgcattca
ttttatgttt caggttcagg gggaggtgtg
ggaggttttt taaagcaagt aaaacctcta
ca

In one embodiment, therefore, the polyA signal sequence comprises a nucleotide sequence as set out in SEQ ID No: 11, or a fragment or variant thereof.

Accordingly, in one embodiment, the rAAV vector comprises a genetic construct comprising, in a 5′ to 3′ direction, a CMV promoter sequence, a first coding sequence encoding TrkB, a nucleotide sequence encoding a linker peptide, a nucleotide sequence encoding a signal peptide, a second coding sequence encoding mBDNF, and a woodchuck hepatitis virus post-transcriptional regulatory element (WPRE).

Accordingly, in another embodiment, the rAAV vector comprises a genetic construct comprising, in a 5′ to 3′ direction, a CMV promoter sequence, a first coding sequence encoding TrkB, a nucleotide sequence encoding a linker peptide, a nucleotide sequence encoding a signal peptide, a second coding sequence encoding mBDNF, a woodchuck hepatitis virus post-transcriptional regulatory element (WPRE), and a simian virus 40 (SV40) polyA signal sequence.

Accordingly, in another embodiment, the rAAV vector comprises a genetic construct comprising, in a 5′ to 3′ direction, a CMV promoter sequence, a first coding sequence encoding TrkB, a nucleotide sequence encoding a linker peptide (in some embodiments, a P2A peptide), a nucleotide sequence encoding a signal peptide, a second coding sequence encoding mBDNF, a woodchuck hepatitis virus post-transcriptional regulatory element (WPRE), and a simian virus 40 (SV40) polyA signal sequence.

In one embodiment, the rAAV vector comprises left and/or right Inverted Terminal Repeat sequences (ITRs). In one embodiment, each ITR is disposed at the 5′ and/or 3′ end of the AAV genome. Herein, “inverted terminal repeats (ITRs)” are sequences that are known to those skilled in the art and refer to sequences that exist at each end of the genomic DNA of AAV and form a hairpin loop. AAV is classified into different serotypes based on the capsid protein sequences, such as AAV1 and AAV2, and AAV genomes of different serotypes contain different ITR sequences. However, an AAV genome containing an ITR derived from one serotype can be packaged into a capsid derived from another serotype. Each ITR may be a wild-type sequence or a variant having the function of an ITR. In one embodiment, each ITR is an ITR derived from any of AAV1, AAV2, AAV3, AAV4, AAV5, AAV8, AAV9, and so on, or a modified ITR therefrom. In some embodiments, each ITR is an AAV2-derived ITR.

One embodiment of the nucleotide sequence encoding the 5′ ITR is referred to herein as SEQ ID No: 12, as follows:

[SEQ ID No: 12]
ctgcgcgctc gctcgctcac tgaggccgcc
cgggcaaagc ccgggcgtcg ggcgaccttt
ggtcgcccgg cctcagtgag cgagcgagcg
cgcagagagg gagtggccaa ctccatcact
aggggttcct

One embodiment of the nucleotide sequence encoding the 3′ ITR is referred to herein as SEQ ID No: 13, as follows:

[SEQ ID No: 13]
aggaacccct agtgatggag ttggccactc
cctctctgcg cgctcgctcg ctcactgagg
ccgggcgacc aaaggtcgcc cgacgcccgg
gctttgcccg ggcggcctca gtgagcgagc
gagcgcgcag

Accordingly, in one embodiment, the 5′ ITR and the 3′ ITR comprise a nucleotide sequence set forth in SEQ ID No: 12 and the complementary sequence to the nucleotide sequence set forth in SEQ ID No: 12 (a nucleotide sequence set forth in SEQ ID NO: 13), respectively.

Accordingly, in one embodiment, the rAAV vector comprises a genetic construct comprising, in a 5′ to 3′ direction, a 5′ ITR, a CMV promoter sequence, a first coding sequence encoding TrkB, a nucleotide sequence encoding a linker peptide (in one embodiment, a P2A peptide), a nucleotide sequence encoding a signal peptide, a second coding sequence encoding mBDNF, a woodchuck hepatitis virus post-transcriptional regulatory element (WPRE), a simian virus 40 (SV40) polyA signal sequence, and a 3′ ITR.

Depending on the promoters, host cells, and so on to be used, the rAAV vector of the presently disclosed subject matter may further comprise various expression regulatory elements (e.g., see Goeddel, Gene Expression Technology, Methods in Enzymology, 1990. 185. Academic Press, San Diego), a translation initiation codon, a translation termination codon, a Kozak sequence, a splicing junction, and so on.

The genetic construct comprised within the rAAV vector of the presently disclosed subject matter can be synthesized by using a standard polynucleotide synthesis method known in the art on the basis of sequence information. A variant of the polynucleotide can be produced through introducing a mutation at a specific site of a given polynucleotide by using a method known to those skilled in the art such as site-specific mutagenesis.

From the foregoing, the skilled person will appreciate the nucleotide sequence of an embodiment of the genetic construct comprised within the rAAV vector of the first aspect, as well as the amino acid sequence of the encoded transgene. However, for the avoidance of doubt, in one embodiment, the rAAV vector of the presently disclosed subject matter is a rAAV vector comprising a genetic construct comprising a nucleotide sequence referred to herein as SEQ ID No: 14, as follows:

[SEQ ID No: 14]
ttaatagtaa tcaattacgg ggtcattagt tcatagccca tatatggagt tccgcgttac
ataacttacg gtaaatggcc cgcctggctg accgcccaac gacccccgcc cattgacgtc
aataatgacg tatgttccca tagtaacgcc aatagggact ttccattgac gtcaatgggt
ggagtattta cggtaaactg cccacttggc agtacatcaa gtgtatcata tgccaagtac
gccccctatt gacgtcaatg acggtaaatg gcccgcctgg cattatgccc agtacatgac
cttatgggac tttcctactt ggcagtacat ctacgtatta gtcatcgcta ttaccatggt
gatgcggttt tggcagtaca tcaatgggcg tggatagcgg tttgactcac ggggatttcc
aagtctccac cccattgacg tcaatgggag tttgttttgg caccaaaatc aacgggactt
tccaaaatgt cgtaacaact ccgccccatt gacgcaaatg ggcggtaggc gtgtacggtg
ggaggtctat ataagcagag ctggtttagt ggatatcctt aagcatgtcg tcctggataa
ggtggcatgg acccgccatg gcgcggctct ggggcttctg ctggctggtt gtgggcttct
ggagggccgc tttcgcctgt cccacgtcct gcaaatgcag tgcctctcgg atctggtgca
gcgacccttc tcctggcatc gtggcatttc cgagattgga gcctaacagt gtagatcctg
agaacatcac cgaaattttc atcgcaaacc agaaaaggtt agaaatcatc aacgaagatg
atgttgaagc ttatgtggga ctgagaaatc tgacaattgt ggattctgga ttaaaatttg
tggctcataa agcatttctg aaaaacagca acctgcagca catcaatttt acccgaaaca
aactgacgag tttgtctagg aaacatttcc gtcaccttga cttgtctgaa ctgatcctgg
tgggcaatcc atttacatgc tcctgtgaca ttatgtggat caagactctc caagaggcta
aatccagtcc agacactcag gatttgtact gcctgaatga aagcagcaag aatattcccc
tggcaaacct gcagataccc aattgtggtt tgccatctgc aaatctggcc gcacctaacc
tcactgtgga ggaaggaaag tctatcacat tatcctgtag tgtggcaggt gatccggttc
ctaatatgta ttgggatgtt ggtaacctgg tttccaaaca tatgaatgaa acaagccaca
cacagggctc cttaaggata actaacattt catccgatga cagtgggaag cagatctctt
gtgtggcgga aaatcttgta ggagaagatc aagattctgt caacctcact gtgcattttg
caccaactat cacatttctc gaatctccaa cctcagacca ccactggtgc attccattca
ctgtgaaagg caaccccaaa ccagcgcttc agtggttcta taacggggca atattgaatg
agtccaaata catctgtact aaaatacatg ttaccaatca cacggagtac cacggctgcc
tccagctgga taatcccact cacatgaaca atggggacta cactctaata gccaagaatg
agtatgggaa ggatgagaaa cagatttctg ctcacttcat gggctggcct ggaattgacg
atggtgcaaa cccaaattat cctgatgtaa tttatgaaga ttatggaact gcagcgaatg
acatcgggga caccacgaac agaagtaatg aaatcccttc cacagacgtc actgataaaa
ccggtcggga acatctctcg gtctatgctg tggtggtgat tgcgtctgtg gtgggatttt
gccttttggt aatgctgttt ctgcttaagt tggcaagaca ctccaagttt ggcatgaaag
gcccagcctc cgttatcagc aatgatgatg actctgccag cccactccat cacatctcca
atgggagtaa cactccatct tcttcggaag gtggcccaga tgctgtcatt attggaatga
ccaagatccc tgtcattgaa aatccccagt actttggcat caccaacagt cagctcaagc
cagacacatt tgttcagcac atcaagcgac ataacattgt tctgaaaagg gagctaggcg
aaggagcctt tggaaaagtg ttcctagctg aatgctataa cctctgtcct gagcaggaca
agatcttggt ggcagtgaag accctgaagg atgccagtga caatgcacgc aaggacttcc
accgtgaggc cgagctcctg accaacctcc agcatgagca catcgtcaag ttctatggcg
tctgcgtgga gggcgacccc ctcatcatgg tctttgagta catgaagcat ggggacctca
acaagttcct cagggcacac ggccctgatg ccgtgctgat ggctgagggc aacccgccca
cggaactgac gcagtcgcag atgctgcata tagcccagca gatcgccgcg ggcatggtct
acctggcgtc ccagcacttc gtgcaccgcg atttggccac caggaactgc ctggtcgggg
agaacttgct ggtgaaaatc ggggactttg ggatgtcccg ggacgtgtac agcactgact
actacagggt cggtggccac acaatgctgc ccattcgctg gatgcctcca gagagcatca
tgtacaggaa attcacgacg gaaagcgacg tctggagcct gggggtcgtg ttgtgggaga
ttttcaccta tggcaaacag ccctggtacc agctgtcaaa caatgaggtg atagagtgta
tcactcaggg ccgagtcctg cagcgacccc gcacgtgccc ccaggaggtg tatgagctga
tgctggggtg ctggcagcga gagccccaca tgaggaagaa catcaagggc atccataccc
tccttcagaa cttggccaag gcatctccgg tctacctgga cattctaggc agcggcgcca
caaatttctc cctgctgaag caggcaggcg acgtggagga gaaccctgga ccaatgcgga
tccttctgct tactatggtt atttcatact ttggttgcat gaaggctcac tctgaccctg
cccgccgagg ggagctgagc gtgtgtgaca gtattagtga gtgggtaacg gcggcagaca
aaaagactgc agtggacatg tcgggcggga cggtcacagt ccttgaaaag gtccctgtat
caaaaggcca actgaagcaa tacttctacg agaccaagtg caatcccatg ggttacacaa
aagaaggctg caggggcata gacaaaaggc attggaactc ccagtgccga actacccagt
cgtacgtgcg ggcccttacc atggatagca aaaagagaat tggctggcga ttcataagga
tagacacttc ttgtgtatgt acattgacca ttaaaagggg aagatag

Accordingly, in one embodiment, the rAAV vector according to the first aspect comprises a genetic construct comprising a nucleotide sequence as set out in SEQ ID No: 14, or a variant or fragment thereof.

The genetic construct consisting of the nucleotide sequence set forth in SEQ ID NO: 14 (hereinafter, also referred to as “CMV-hTrkB-P2A-mSP-hmBDNF”) comprises, from 5′ to 3′ direction, a CMV promoter sequence consisting of the nucleotide sequence set forth in SEQ ID NO: 1, a nucleotide sequence encoding TrkB and consisting of the nucleotide sequence set forth in SEQ ID NO: 3, a nucleotide sequence encoding an SG-P2A peptide and consisting of the nucleotide sequence set forth in SEQ ID NO: 9, a nucleotide sequence encoding a signal peptide and consisting of the nucleotide sequence set forth in SEQ ID NO: 7, and a nucleotide sequence encoding mBDNF and consisting of the nucleotide sequence set forth in SEQ ID NO: 5, in the order presented.

In another embodiment, the rAAV vector of the presently disclosed subject matter is a rAAV vector comprising a genetic construct comprising a nucleotide sequence referred to here as SEQ ID No: 15, as follows:

[SEQ ID No: 15]
ttaatagtaa tcaattacgg ggtcattagt tcatagccca tatatggagt tccgcgttac
ataacttacg gtaaatggcc cgcctggctg accgcccaac gacccccgcc cattgacgtc
aataatgacg tatgttccca tagtaacgcc aatagggact ttccattgac gtcaatgggt
ggagtattta cggtaaactg cccacttggc agtacatcaa gtgtatcata tgccaagtac
gccccctatt gacgtcaatg acggtaaatg gcccgcctgg cattatgccc agtacatgac
cttatgggac tttcctactt ggcagtacat ctacgtatta gtcatcgcta ttaccatggt
gatgcggttt tggcagtaca tcaatgggcg tggatagcgg tttgactcac ggggatttcc
aagtctccac cccattgacg tcaatgggag tttgttttgg caccaaaatc aacgggactt
tccaaaatgt cgtaacaact ccgccccatt gacgcaaatg ggcggtaggc gtgtacggtg
ggaggtctat ataagcagag ctggtttagt ggatatcctt aagcatgtcg tcctggataa
ggtggcatgg acccgccatg gcgcggctct ggggcttctg ctggctggtt gtgggcttct
ggagggccgc tttcgcctgt cccacgtcct gcaaatgcag tgcctctcgg atctggtgca
gcgacccttc tectggcatc gtggcatttc cgagattgga gcctaacagt gtagatcctg
agaacatcac cgaaattttc atcgcaaacc agaaaaggtt agaaatcatc aacgaagatg
atgttgaagc ttatgtggga ctgagaaatc tgacaattgt ggattctgga ttaaaatttg
tggctcataa agcatttctg aaaaacagca acctgcagca catcaatttt acccgaaaca
aactgacgag tttgtctagg aaacatttcc gtcaccttga cttgtctgaa ctgatcctgg
tgggcaatcc atttacatgc tcctgtgaca ttatgtggat caagactctc caagaggcta
aatccagtcc agacactcag gatttgtact gcctgaatga aagcagcaag aatattcccc
tggcaaacct gcagataccc aattgtggtt tgccatctgc aaatctggcc gcacctaacc
tcactgtgga ggaaggaaag tctatcacat tatcctgtag tgtggcaggt gatccggttc
ctaatatgta ttgggatgtt ggtaacctgg tttccaaaca tatgaatgaa acaagccaca
cacagggctc cttaaggata actaacattt catccgatga cagtgggaag cagatctctt
gtgtggcgga aaatcttgta ggagaagatc aagattctgt caacctcact gtgcattttg
caccaactat cacatttctc gaatctccaa cctcagacca ccactggtgc attccattca
ctgtgaaagg caaccccaaa ccagcgcttc agtggttcta taacggggca atattgaatg
agtccaaata catctgtact aaaatacatg ttaccaatca cacggagtac cacggctgcc
tccagctgga taatcccact cacatgaaca atggggacta cactctaata gccaagaatg
agtatgggaa ggatgagaaa cagatttctg ctcacttcat gggctggcct ggaattgacg
atggtgcaaa cccaaattat cctgatgtaa tttatgaaga ttatggaact gcagcgaatg
acatcgggga caccacgaac agaagtaatg aaatcccttc cacagacgtc actgataaaa
ccggtcggga acatctctcg gtctatgctg tggtggtgat tgcgtctgtg gtgggatttt
gccttttggt aatgctgttt ctgcttaagt tggcaagaca ctccaagttt ggcatgaaag
gcccagcctc cgttatcagc aatgatgatg actctgccag cccactccat cacatctcca
atgggagtaa cactccatct tcttcggaag gtggcccaga tgctgtcatt attggaatga
ccaagatccc tgtcattgaa aatccccagt actttggcat caccaacagt cagctcaagc
cagacacatt tgttcagcac atcaagcgac ataacattgt tctgaaaagg gagctaggcg
aaggagcctt tggaaaagtg ttcctagctg aatgctataa cctctgtcct gagcaggaca
agatcttggt ggcagtgaag accctgaagg atgccagtga caatgcacgc aaggacttcc
accgtgaggc cgagctcctg accaacctcc agcatgagca catcgtcaag ttctatggcg
tctgcgtgga gggcgacccc ctcatcatgg totttgagta catgaagcat ggggacctca
acaagttcct cagggcacac ggccctgatg ccgtgctgat ggctgagggc aacccgccca
cggaactgac gcagtcgcag atgctgcata tagcccagca gatcgccgcg ggcatggtct
acctggcgtc ccagcacttc gtgcaccgcg atttggccac caggaactgc ctggtcgggg
agaacttgct ggtgaaaatc ggggactttg ggatgtcccg ggacgtgtac agcactgact
actacagggt cggtggccac acaatgctgc ccattcgctg gatgcctcca gagagcatca
tgtacaggaa attcacgacg gaaagcgacg tctggagcct gggggtcgtg ttgtgggaga
ttttcaccta tggcaaacag ccctggtacc agctgtcaaa caatgaggtg atagagtgta
tcactcaggg ccgagtcctg cagcgacccc gcacgtgccc ccaggaggtg tatgagctga
tgctggggtg ctggcagcga gagccccaca tgaggaagaa catcaagggc atccataccc
tccttcagaa cttggccaag gcatctccgg tctacctgga cattctaggc agcggcgcca
caaatttctc cctgctgaag caggcaggcg acgtggagga gaaccctgga ccaatgcgga
tccttctgct tactatggtt atttcatact ttggttgcat gaaggctcac tctgaccctg
cccgccgagg ggagctgagc gtgtgtgaca gtattagtga gtgggtaacg gcggcagaca
aaaagactgc agtggacatg tcgggcggga cggtcacagt ccttgaaaag gtccctgtat
caaaaggcca actgaagcaa tacttctacg agaccaagtg caatcccatg ggttacacaa
aagaaggctg caggggcata gacaaaaggc attggaactc ccagtgccga actacccagt
cgtacgtgcg ggcccttacc atggatagca aaaagagaat tggctggcga ttcataagga
tagacacttc ttgtgtatgt acattgacca ttaaaagggg aagatagtat actactagta
cgcggccgca ccggtgtaca atcaacctct ggattacaaa atttgtgaaa gattgactgg
tattcttaac tatgttgctc cttttacgct atgtggatac gctgctttaa tgcctttgta
tcatgctatt gcttcccgta tggctttcat tttctcctcc ttgtataaat cctggttagt
tcttgccacg gcggaactca tcgccgcctg ccttgcccgc tgctggacag gggctcggct
gttgggcact gacaattccg tggtgtgaat tcgagctagg tacagcttat cgataccgtc
gacagcagac atgataagat acattgatga gtttggacaa accacaacta gaatgcagtg
aaaaaaatgc tttatttgtg aaatttgtga tgctattgct ttatttgtaa ccattataag
ctgcaataaa caagttaaca acaacaattg cattcatttt atgtttcagg ttcaggggga
ggtgtgggag gttttttaaa gcaagtaaaa cctctacaaa tgtggtatg

Accordingly, in another embodiment, the rAAV vector according to the first aspect comprises a genetic construct comprising a nucleotide sequence as set out in SEQ ID No: 15, or a variant or fragment thereof.

Here, the genetic construct consisting of the nucleotide sequence set forth in SEQ ID NO: 15 (hereinafter, also referred to as “CMV-hTrkB-P2A-mSP-hmBDNF-WPRE(S)-SV40 pA”) is a genetic construct comprising, from 5′ to 3′ direction, a CMV promoter sequence consisting of the nucleotide sequence set forth in SEQ ID NO: 1, a nucleotide sequence encoding TrkB and consisting of the nucleotide sequence set forth in SEQ ID NO: 3, a nucleotide sequence encoding an SG-P2A peptide and consisting of the nucleotide sequence set forth in SEQ ID NO: 9, a nucleotide sequence encoding a signal peptide and consisting of the nucleotide sequence set forth in SEQ ID NO: 7, a nucleotide sequence encoding mBDNF and consisting of the nucleotide sequence set forth in SEQ ID NO: 5, a WPRE consisting of the nucleotide sequence set forth in SEQ ID NO: 10, and an SV40 polyA signal sequence consisting of the nucleotide sequence set forth in SEQ ID NO: 11, in the order presented.

In another embodiment, the rAAV vector of the presently disclosed subject matter is a rAAV vector comprising a genetic construct comprising a nucleotide sequence referred to here as SEQ ID No: 16, as follows:

[SEQ ID No: 16]
ctgcgcgctc gctcgctcac tgaggccgcc cgggcaaagc ccgggcgtcg ggcgaccttt
ggtcgcccgg cctcagtgag cgagcgagcg cgcagagagg gagtggccaa ctccatcact
aggggttcct atcgatatca agctttgtag ttaatgatta acccgccatg ctacttatct
acgtagccat gctctagtat cgatatcaag ctttaatagt aatcaattac ggggtcatta
gttcatagcc catatatgga gttccgcgtt acataactta cggtaaatgg cccgcctggc
tgaccgccca acgacccccg cccattgacg tcaataatga cgtatgttcc catagtaacg
ccaataggga ctttccattg acgtcaatgg gtggagtatt tacggtaaac tgcccacttg
gcagtacatc aagtgtatca tatgccaagt acgcccccta ttgacgtcaa tgacggtaaa
tggcccgcct ggcattatgc ccagtacatg accttatggg actttcctac ttggcagtac
atctacgtat tagtcatcgc tattaccatg gtgatgcggt tttggcagta catcaatggg
cgtggatagc ggtttgactc acggggattt ccaagtctcc accccattga cgtcaatggg
agtttgtttt ggcaccaaaa tcaacgggac tttccaaaat gtcgtaacaa ctccgcccca
ttgacgcaaa tgggcggtag gcgtgtacgg tgggaggtct atataagcag agctggttta
gtggatatcc ttaagcatgt cgtcctggat aaggtggcat ggacccgcca tggcgcggct
ctggggcttc tgctggctgg ttgtgggctt ctggagggcc gctttcgcct gtcccacgtc
ctgcaaatgc agtgcctctc ggatctggtg cagcgaccct tctcctggca tcgtggcatt
tccgagattg gagcctaaca gtgtagatcc tgagaacatc accgaaattt tcatcgcaaa
ccagaaaagg ttagaaatca tcaacgaaga tgatgttgaa gcttatgtgg gactgagaaa
tctgacaatt gtggattctg gattaaaatt tgtggctcat aaagcatttc tgaaaaacag
caacctgcag cacatcaatt ttacccgaaa caaactgacg agtttgtcta ggaaacattt
ccgtcacctt gacttgtctg aactgatcct ggtgggcaat ccatttacat gctcctgtga
cattatgtgg atcaagactc tccaagaggc taaatccagt ccagacactc aggatttgta
ctgcctgaat gaaagcagca agaatattcc cctggcaaac ctgcagatac ccaattgtgg
tttgccatct gcaaatctgg ccgcacctaa cctcactgtg gaggaaggaa agtctatcac
attatcctgt agtgtggcag gtgatccggt tcctaatatg tattgggatg ttggtaacct
ggtttccaaa catatgaatg aaacaagcca cacacagggc tccttaagga taactaacat
ttcatccgat gacagtggga agcagatctc ttgtgtggcg gaaaatcttg taggagaaga
tcaagattct gtcaacctca ctgtgcattt tgcaccaact atcacatttc tcgaatctcc
aacctcagac caccactggt gcattccatt cactgtgaaa ggcaacccca aaccagcgct
tcagtggttc tataacgggg caatattgaa tgagtccaaa tacatctgta ctaaaataca
tgttaccaat cacacggagt accacggctg cctccagctg gataatccca ctcacatgaa
caatggggac tacactctaa tagccaagaa tgagtatggg aaggatgaga aacagatttc
tgctcacttc atgggctggc ctggaattga cgatggtgca aacccaaatt atcctgatgt
aatttatgaa gattatggaa ctgcagcgaa tgacatcggg gacaccacga acagaagtaa
tgaaatccct tccacagacg tcactgataa aaccggtcgg gaacatctct cggtctatgc
tgtggtggtg attgcgtctg tggtgggatt ttgccttttg gtaatgctgt ttctgcttaa
gttggcaaga cactccaagt ttggcatgaa aggcccagcc tccgttatca gcaatgatga
tgactctgcc agcccactcc atcacatctc caatgggagt aacactccat cttcttcgga
aggtggccca gatgctgtca ttattggaat gaccaagatc cctgtcattg aaaatcccca
gtactttggc atcaccaaca gtcagctcaa gccagacaca tttgttcagc acatcaagcg
acataacatt gttctgaaaa gggagctagg cgaaggagcc tttggaaaag tgttcctagc
tgaatgctat aacctctgtc ctgagcagga caagatcttg gtggcagtga agaccctgaa
ggatgccagt gacaatgcac gcaaggactt ccaccgtgag gccgagctcc tgaccaacct
ccagcatgag cacatcgtca agttctatgg cgtctgcgtg gagggcgacc ccctcatcat
ggtctttgag tacatgaagc atggggacct caacaagttc ctcagggcac acggccctga
tgccgtgctg atggctgagg gcaacccgcc cacggaactg acgcagtcgc agatgctgca
tatagcccag cagatcgccg cgggcatggt ctacctggcg tcccagcact tcgtgcaccg
cgatttggcc accaggaact gcctggtcgg ggagaacttg ctggtgaaaa tcggggactt
tgggatgtcc cgggacgtgt acagcactga ctactacagg gtcggtggcc acacaatgct
gcccattcgc tggatgcctc cagagagcat catgtacagg aaattcacga cggaaagcga
cgtctggagc ctgggggtcg tgttgtggga gattttcacc tatggcaaac agccctggta
ccagctgtca aacaatgagg tgatagagtg tatcactcag ggccgagtcc tgcagcgacc
ccgcacgtgc ccccaggagg tgtatgagct gatgctgggg tgctggcagc gagagcccca
catgaggaag aacatcaagg gcatccatac cctccttcag aacttggcca aggcatctcc
ggtctacctg gacattctag gcagcggcgc cacaaatttc tccctgctga agcaggcagg
cgacgtggag gagaaccctg gaccaatgcg gatccttctg cttactatgg ttatttcata
ctttggttgc atgaaggctc actctgaccc tgcccgccga ggggagctga gcgtgtgtga
cagtattagt gagtgggtaa cggcggcaga caaaaagact gcagtggaca tgtcgggcgg
gacggtcaca gtccttgaaa aggtccctgt atcaaaaggc caactgaagc aatacttcta
cgagaccaag tgcaatccca tgggttacac aaaagaaggc tgcaggggca tagacaaaag
gcattggaac toccagtgcc gaactaccca gtcgtacgtg cgggccctta ccatggatag
caaaaagaga attggctggc gattcataag gatagacact tottgtgtat gtacattgac
cattaaaagg ggaagatagt atactactag tacgcggccg caccggtgta caatcaacct
ctggattaca aaatttgtga aagattgact ggtattctta actatgttgc tccttttacg
ctatgtggat acgctgcttt aatgcctttg tatcatgcta ttgcttcccg tatggctttc
attttctcct ccttgtataa atcctggtta gttcttgcca cggcggaact catcgccgcc
tgccttgccc gctgctggac aggggctcgg ctgttgggca ctgacaattc cgtggtgtga
attcgagcta ggtacagctt atcgataccg tcgacagcag acatgataag atacattgat
gagtttggac aaaccacaac tagaatgcag tgaaaaaaat gctttatttg tgaaatttgt
gatgctattg ctttatttgt aaccattata agctgcaata aacaagttaa caacaacaat
tgcattcatt ttatgtttca ggttcagggg gaggtgtggg aggtttttta aagcaagtaa
aacctctaca aatgtggtat gctcgagggc atgcaacaac aacaattgca ttcatgaggt
tttttaaagc aagtaaaacc tctacaaatg tggtaaaatc cgataaggac tagagcatgg
ctacgtagat aagtagcatg gcgggttaat cattaactac aagatctagg aacccctagt
gatggagttg gccactccct ctctgcgcgc tcgctcgctc actgaggccg ggcgaccaaa
ggtcgcccga cgcccgggct ttgcccgggc ggcctcagtg agcgagcgag cgcgcag

Accordingly, in another embodiment, the rAAV vector according to the first aspect comprises a genetic construct comprising a nucleotide sequence as set out in SEQ ID No: 16, or a variant or fragment thereof.

The genetic construct consisting of the nucleotide sequence set forth in SEQ ID NO: 16 (hereinafter, also referred to as “ITR-CMV-hTrkB-P2A-mSP-hmBDNF-WPRE(S)-SV40 pA-ITR”) is a genetic construct comprising “CMV-hTrkB-P2A-mSP-hmBDNF-WPRE(S)-SV40 pA” (SEQ ID NO: 15) provided with AAV2-derived ITRs on the 5′ side and 3′ side thereof (the nucleotide sequence set forth in SEQ ID NO: 12 and the nucleotide sequence set forth in SEQ ID NO: 13, respectively).

In another embodiment, the rAAV vector of the presently disclosed subject matter is a rAAV vector comprising a genetic construct comprising a nucleotide sequence referred to here as SEQ ID NO: 17, as follows:

[SEQ ID No: 17]
ctgcgcgctc gctcgctcac tgaggccgcc cgggcaaagc ccgggcgtcg ggcgaccttt
ggtcgcccgg cctcagtgag cgagcgagcg cgcagagagg gagtggccaa ctccatcact
aggggttcct atcgatatca agctttaata gtaatcaatt acggggtcat tagttcatag
cccatatatg gagttccgcg ttacataact tacggtaaat ggcccgcctg gctgaccgcc
caacgacccc cgcccattga cgtcaataat gacgtatgtt cccatagtaa cgccaatagg
gactttccat tgacgtcaat gggtggagta tttacggtaa actgcccact tggcagtaca
tcaagtgtat catatgccaa gtacgccccc tattgacgtc aatgacggta aatggcccgc
ctggcattat gcccagtaca tgaccttatg ggactttcct acttggcagt acatctacgt
attagtcatc gctattacca tggtgatgcg gttttggcag tacatcaatg ggcgtggata
gcggtttgac tcacggggat ttccaagtct ccaccccatt gacgtcaatg ggagtttgtt
ttggcaccaa aatcaacggg actttccaaa atgtcgtaac aactccgccc cattgacgca
aatgggcggt aggcgtgtac ggtgggaggt ctatataagc agagctggtt tagtggatat
ccttaagcat gtcgtcctgg ataaggtggc atggacccgc catggcgcgg ctctggggct
tctgctggct ggttgtgggc ttctggaggg ccgctttcgc ctgtcccacg tcctgcaaat
gcagtgcctc tcggatctgg tgcagcgacc cttctcctgg catcgtggca tttccgagat
tggagcctaa cagtgtagat cctgagaaca tcaccgaaat tttcatcgca aaccagaaaa
ggttagaaat catcaacgaa gatgatgttg aagcttatgt gggactgaga aatctgacaa
ttgtggattc tggattaaaa tttgtggctc ataaagcatt tctgaaaaac agcaacctgc
agcacatcaa ttttacccga aacaaactga cgagtttgtc taggaaacat ttccgtcacc
ttgacttgtc tgaactgatc ctggtgggca atccatttac atgctcctgt gacattatgt
ggatcaagac tctccaagag gctaaatcca gtccagacac tcaggatttg tactgcctga
atgaaagcag caagaatatt cccctggcaa acctgcagat acccaattgt ggtttgccat
ctgcaaatct ggccgcacct aacctcactg tggaggaagg aaagtctatc acattatcct
gtagtgtggc aggtgatccg gttcctaata tgtattggga tgttggtaac ctggtttcca
aacatatgaa tgaaacaagc cacacacagg gctccttaag gataactaac atttcatccg
atgacagtgg gaagcagatc tottgtgtgg cggaaaatct tgtaggagaa gatcaagatt
ctgtcaacct cactgtgcat tttgcaccaa ctatcacatt tctcgaatct ccaacctcag
accaccactg gtgcattcca ttcactgtga aaggcaaccc caaaccagcg cttcagtggt
tctataacgg ggcaatattg aatgagtcca aatacatctg tactaaaata catgttacca
atcacacgga gtaccacggc tgcctccagc tggataatcc cactcacatg aacaatgggg
actacactct aatagccaag aatgagtatg ggaaggatga gaaacagatt tctgctcact
tcatgggctg gcctggaatt gacgatggtg caaacccaaa ttatcctgat gtaatttatg
aagattatgg aactgcagcg aatgacatcg gggacaccac gaacagaagt aatgaaatcc
cttccacaga cgtcactgat aaaaccggtc gggaacatct ctcggtctat gctgtggtgg
tgattgcgtc tgtggtggga ttttgccttt tggtaatgct gtttctgctt aagttggcaa
gacactccaa gtttggcatg aaaggcccag cctccgttat cagcaatgat gatgactctg
ccagcccact ccatcacatc tccaatggga gtaacactcc atcttcttcg gaaggtggcc
cagatgctgt cattattgga atgaccaaga tccctgtcat tgaaaatccc cagtactttg
gcatcaccaa cagtcagctc aagccagaca catttgttca gcacatcaag cgacataaca
ttgttctgaa aagggagcta ggcgaaggag cctttggaaa agtgttccta gctgaatgct
ataacctctg tcctgagcag gacaagatct tggtggcagt gaagaccctg aaggatgcca
gtgacaatgc acgcaaggac ttccaccgtg aggccgagct cctgaccaac ctccagcatg
agcacatcgt caagttctat ggcgtctgcg tggagggcga ccccctcatc atggtctttg
agtacatgaa gcatggggac ctcaacaagt tcctcagggc acacggccct gatgccgtgc
tgatggctga gggcaacccg cccacggaac tgacgcagtc gcagatgctg catatagccc
agcagatcgc cgcgggcatg gtctacctgg cgtcccagca cttcgtgcac cgcgatttgg
ccaccaggaa ctgcctggtc ggggagaact tgctggtgaa aatcggggac tttgggatgt
cccgggacgt gtacagcact gactactaca gggtcggtgg ccacacaatg ctgcccattc
gctggatgcc tccagagagc atcatgtaca ggaaattcac gacggaaagc gacgtctgga
gcctgggggt cgtgttgtgg gagattttca cctatggcaa acagccctgg taccagctgt
caaacaatga ggtgatagag tgtatcactc agggccgagt cctgcagcga ccccgcacgt
gcccccagga ggtgtatgag ctgatgctgg ggtgctggca gcgagagccc cacatgagga
agaacatcaa gggcatccat accctccttc agaacttggc caaggcatct ccggtctacc
tggacattct aggcagcggc gccacaaatt tctccctgct gaagcaggca ggcgacgtgg
aggagaaccc tggaccaatg cggatccttc tgcttactat ggttatttca tactttggtt
gcatgaaggc tcactctgac cctgcccgcc gaggggagct gagcgtgtgt gacagtatta
gtgagtgggt aacggcggca gacaaaaaga ctgcagtgga catgtcgggc gggacggtca
cagtccttga aaaggtccct gtatcaaaag gccaactgaa gcaatacttc tacgagacca
agtgcaatcc catgggttac acaaaagaag gctgcagggg catagacaaa aggcattgga
actcccagtg ccgaactacc cagtcgtacg tgcgggccct taccatggat agcaaaaaga
gaattggctg gcgattcata aggatagaca cttcttgtgt atgtacattg accattaaaa
ggggaagata gtatactact agtacgcggc cgcaccggtg tacaatcaac ctctggatta
caaaatttgt gaaagattga ctggtattct taactatgtt gctcctttta cgctatgtgg
atacgctgct ttaatgcctt tgtatcatgc tattgcttcc cgtatggctt tcattttctc
ctccttgtat aaatcctggt tagttcttgc cacggcggaa ctcatcgccg cctgccttgc
ccgctgctgg acaggggctc ggctgttggg cactgacaat tccgtggtgt gaattcgagc
taggtacagc ttatcgatac cgtcgacagc agacatgata agatacattg atgagtttgg
acaaaccaca actagaatgc agtgaaaaaa atgctttatt tgtgaaattt gtgatgctat
tgctttattt gtaaccatta taagctgcaa taaacaagtt aacaacaaca attgcattca
ttttatgttt caggttcagg gggaggtgtg ggaggttttt taaagcaagt aaaacctcta
caaatgtggt atgctcgagg gcatgcaaca acaacaattg cattcatgag gttttttaaa
gcaagtaaaa cctctacaaa tgtggtaaaa tccgataagg actagagcat ggctacgtag
ataagtagca tggcgggtta atcattaact acaagatcta ggaaccccta gtgatggagt
tggccactcc ctctctgcgc gctcgctcgc tcactgaggc cgggcgacca aaggtcgccc
gacgcccggg ctttgcccgg gcggcctcag tgagcgagcg agcgcgcag

Accordingly, in another embodiment, the rAAV vector according to the first aspect comprises a genetic construct comprising a nucleotide sequence as set out in SEQ ID No: 17, or a variant or fragment thereof.

The genetic construct consisting of the nucleotide sequence set forth in SEQ ID NO: 17 (hereinafter, also referred to as “ITR-CMV-hTrkB-P2A-mSP-hmBDNF-WPRE(S)-SV40 pA-ITR (2)”) is a genetic construct that comprises “CMV-hTrkB-P2A-mSP-hmBDNF-WPRE(S)-SV40 pA” (SEQ ID NO: 15) provided with AAV2-derived ITRs on the 5′ side and 3′ side thereof (the nucleotide sequence set forth in SEQ ID NO: 12 and the nucleotide sequence set forth in SEQ ID NO: 13, respectively) and is different from SEQ ID NO: 16 in the nucleotide sequence between the 5′ ITR and the CMV promoter.

Any serotype of AAV that allows TrkB and BDNF to be expressed in host cells is applicable, without limitation, in the presently disclosed subject matter, and AAV1, AAV2, AAV3, AAV4, AAV5, AAV8, AAV9, rAAV2.7m8 vector, rAAV2 Max vector, and so on can be used. Herein, the rAAV vector of the presently disclosed subject matter derived from any of the mentioned AAV serotypes is referred to as a rAAV1 vector, rAAV2 vector, rAAV2.7m8 vector, rAAV2 Max vector, rAAV3 vector, rAAV4 vector, rAAV5 vector, rAAV8 vector, or rAAV9 vector. The rAAV vector in the presently disclosed subject matter may be a modified rAAV vector in which the amino acid sequence of the capsid protein is modified.

In some embodiments, the rAAV vector of the presently disclosed subject matter is a rAAV2 vector. In one embodiment, the rAAV vector is a rAAV2.7m8 vector. The rAAV2.7m8 vector comprises a retina-specific 7m8 peptide insertion between amino acids 587 and 588 (N587_R588insLALGETTRPA), and has been shown to exhibit improved photoreceptor transduction following intravitreal injection compared to unmodified rAAV2. See WO2012/145601 and Reid et al., 2017. Improvement of photoreceptor targeting via intravitreal delivery in mouse and human retina using combinatory rAAV2 capsid mutant vectors. Investigative ophthalmology & visual science, 58(14), pp. 6429-6439.

One embodiment of the amino acid sequence of the capsid of the rAAV2.7m8 vector is provided here as SEQ ID No: 18, as follows:

[SEQ ID No: 18]
MAADGYLPDWLEDTLSEGIRQWWKLKPGPPPPKPAERHKDDSRGL
VLPGYKYLGPENGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDOYLYYLSRTNT
PSGTTTQSRLOFSQAGASDIRDOSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GNLALGETTRPARQAATADVNTQGVLPGMVWODRDVYLOGPIWAK
IPHTDGHFHPSPLMGGFGLKHPPPQILIKNTPVPANPSTTFSAAK
FASFITQYSTGQVSVEIEWELQKENSKRWNPEIQYTSNYNKSVNV
DFTVDTNGVYSEPRPIGTRYLTRNL*

One embodiment of the nucleotide sequence encoding the Cap gene for the rAAV2.7m8 vector is provided here as SEQ ID No: 22, as follows:

[SEQ ID No: 22]
ATGGCTGCCGATGGTTATCTTCCAGATTGGCTCGAGGACACTCTC
TCTGAAGGAATAAGACAGTGGTGGAAGCTCAAACCTGGCCCACCA
CCACCAAAGCCCGCAGAGCGGCATAAGGACGACAGCAGGGGTCTT
GTGCTTCCTGGGTACAAGTACCTCGGACCCTTCAACGGACTCGAC
AAGGGAGAGCCGGTCAACGAGGCAGACGCCGCGGCCCTCGAGCAC
GACAAAGCCTACGACCGGCAGCTCGACAGCGGAGACAACCCGTAC
CTCAAGTACAACCACGCCGACGCGGAGTTTCAGGAGCGCCTTAAA
GAAGATACGTCTTTTGGGGGCAACCTCGGACGAGCAGTCTTCCAG
GCGAAAAAGAGGGTTCTTGAACCTCTGGGCCTGGTTGAGGAACCT
GTTAAGACGGCTCCGGGAAAAAAGAGGCCGGTAGAGCACTCTCCT
GTGGAGCCAGACTCCTCCTCGGGAACCGGAAAGGCGGGCCAGCAG
CCTGCAAGAAAAAGATTGAATTTTGGTCAGACTGGAGACGCAGAC
TCAGTACCTGACCCCCAGCCTCTCGGACAGCCACCAGCAGCCCCC
TCTGGTCTGGGAACTAATACGATGGCTACAGGCAGTGGCGCACCA
ATGGCAGACAATAACGAGGGCGCCGACGGAGTGGGTAATTCCTCG
GGAAATTGGCATTGCGATTCCACATGGATGGGCGACAGAGTCATC
ACCACCAGCACCCGAACCTGGGCCCTGCCCACCTACAACAACCAC
CTCTACAAACAAATTTCCAGCCAATCAGGAGCCTCGAACGACAAT
CACTACTTTGGCTACAGCACCCCTTGGGGGTATTTTGACTTCAAC
AGATTCCACTGCCACTTTTCACCACGTGACTGGCAAAGACTCATC
AACAACAACTGGGGATTCCGACCCAAGAGACTCAACTTCAAGCTC
TTTAACATTCAAGTCAAAGAGGTCACGCAGAATGACGGTACGACG
ACGATTGCCAATAACCTTACCAGCACGGTTCAGGTGTTTACTGAC
TCGGAGTACCAGCTCCCGTACGTCCTCGGCTCGGCGCATCAAGGA
TGCCTCCCGCCGTTCCCAGCAGACGTCTTCATGGTGCCACAGTAT
GGATACCTCACCCTGAACAACGGGAGTCAGGCAGTAGGACGCTCT
TCATTTTACTGCCTGGAGTACTTTCCTTCTCAGATGCTGCGTACC
GGAAACAACTTTACCTTCAGCTACACTTTTGAGGACGTTCCTTTC
CACAGCAGCTACGCTCACAGCCAGAGTCTGGACCGTCTCATGAAT
CCTCTCATCGACCAGTACCTGTATTACTTGAGCAGAACAAACACT
CCAAGTGGAACCACCACGCAGTCAAGGCTTCAGTTTTCTCAGGCC
GGAGCGAGTGACATTCGGGACCAGTCTAGGAACTGGCTTCCTGGA
CCCTGTTACCGCCAGCAGCGAGTATCAAAGACATCTGCGGATAAC
AACAACAGTGAATACTCGTGGACTGGAGCTACCAAGTACCACCTC
AATGGCAGAGACTCTCTGGTGAATCCGGGCCCGGCCATGGCAAGC
CACAAGGACGATGAAGAAAAGTTTTTTCCTCAGAGCGGGGTTCTC
ATCTTTGGGAAGCAAGGCTCAGAGAAAACAAATGTGGACATTGAA
AAGGTCATGATTACAGACGAAGAGGAAATCAGGACAACCAATCCC
GTGGCTACGGAGCAGTATGGTTCTGTATCTACCAACCTCCAGAGA
GGCAACctagcactcggcgaaacaacaagacctgctAGACAAGCA
GCTACCGCAGATGTCAACACACAAGGCGTTCTTCCAGGCATGGTC
TGGCAGGACAGAGATGTGTACCTTCAGGGGCCCATCTGGGCAAAG
ATTCCACACACGGACGGACATTTTCACCCCTCTCCCCTCATGGGT
GGATTCGGACTTAAACACCCTCCTCCACAGATTCTCATCAAGAAC
ACCCCGGTACCTGCGAATCCTTCGACCACCTTCAGTGCGGCAAAG
TTTGCTTCCTTCATCACACAGTACTCCACGGGACAGGTCAGCGTG
GAGATCGAGTGGGAGCTGCAGAAGGAAAACAGCAAACGCTGGAAT
CCCGAAATTCAGTACACTTCCAACTACAACAAGTCTGTTAATGTG
GACTTTACTGTGGACACTAATGGCGTGTATTCAGAGCCTCGCCCC
ATTGGCACCAGATACCTGACTCGTAATCTGTAA

In another embodiment, the rAAV vector is a rAAV2 Max vector. The rAAV2 Max vector comprises five point mutations: Y272F; Y444F; Y500F; Y730F; and T491V (derived from rAAV2 [QuadYF+TV; see WO2008/124724, WO2013/173512 and WO2015/126972), and a peptide insertion, N587_R588insLALGETTRPA (derived from rAAV2.7m8), and has been shown to demonstrate high levels of transduction. See Reid et al., 2017. Improvement of photoreceptor targeting via intravitreal delivery in mouse and human retina using combinatory rAAV2 capsid mutant vectors. Investigative ophthalmology & visual science, 58(14), pp. 6429-6439.

One embodiment of the amino acid sequence encoding a capsid of the rAAV2 Max vector is provided here as SEQ ID No: 19, as follows:

[SEQ ID No: 19]
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HFFGYSTPWGYFDFNRFHCHFSPRDWORLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYFLSRTNT
PSGTTTOSRLQFSQAGASDIRDOSRNWLPGPCYRQQRVSKVSADN
NNSEFSWTGATKYHLNGRDSLVNPGPAMASHKDDEEKFFPQSGVL
IFGKOGSEKTNVDIEKVMITDEEEIRTTNPVATEQYGSVSTNLQR
GNLALGETTRPARQAATADVNTQGVLPGMVWODRDVYLQGPIWAK
IPHTDGHFHPSPLMGGFGLKHPPPQILIKNTPVPANPSTTFSAAK
FASFITQYSTGQVSVEIEWELQKENSKRWNPEIQYTSNYNKSVNV
DFTVDTNGVYSEPRPIGTRFLTRNL*

One embodiment of the nucleotide sequence encoding the Cap gene for the rAAV2 Max vector is provided here as SEQ ID No: 23, as follows:

[SEQ ID No: 23]
ATGGCTGCCGATGGTTATCTTCCAGATTGGCTCGAGGACACTCTC
TCTGAAGGAATAAGACAGTGGTGGAAGCTCAAACCTGGCCCACCA
CCACCAAAGCCCGCAGAGCGGCATAAGGACGACAGCAGGGGTCTT
GTGCTTCCTGGGTACAAGTACCTCGGACCCTTCAACGGACTCGAC
AAGGGAGAGCCGGTCAACGAGGCAGACGCCGCGGCCCTCGAGCAC
GACAAAGCCTACGACCGGCAGCTCGACAGCGGAGACAACCCGTAC
CTCAAGTACAACCACGCCGACGCGGAGTTTCAGGAGCGCCTTAAA
GAAGATACGTCTTTTGGGGGCAACCTCGGACGAGCAGTCTTCCAG
GCGAAAAAGAGGGTTCTTGAACCTCTGGGCCTGGTTGAGGAACCT
GTTAAGACGGCTCCGGGAAAAAAGAGGCCGGTAGAGCACTCTCCT
GTGGAGCCAGACTCCTCCTCGGGAACCGGAAAGGCGGGCCAGCAG
CCTGCAAGAAAAAGATTGAATTTTGGTCAGACTGGAGACGCAGAC
TCAGTACCTGACCCCCAGCCTCTCGGACAGCCACCAGCAGCCCCC
TCTGGTCTGGGAACTAATACGATGGCTACAGGCAGTGGCGCACCA
ATGGCAGACAATAACGAGGGCGCCGACGGAGTGGGTAATTCCTCG
GGAAATTGGCATTGCGATTCCACATGGATGGGCGACAGAGTCATC
ACCACCAGCACCCGAACCTGGGCCCTGCCCACCTACAACAACCAC
CTCTACAAACAAATTTCCAGCCAATCAGGAGCCTCGAACGACAAT
CACttcTTTGGCTACAGCACCCCTTGGGGGTATTTTGACTTCAAC
AGATTCCACTGCCACTTTTCACCACGTGACTGGCAAAGACTCATC
AACAACAACTGGGGATTCCGACCCAAGAGACTCAACTTCAAGCTC
TTTAACATTCAAGTCAAAGAGGTCACGCAGAATGACGGTACGACG
ACGATTGCCAATAACCTTACCAGCACGGTTCAGGTGTTTACTGAC
TCGGAGTACCAGCTCCCGTACGTCCTCGGCTCGGCGCATCAAGGA
TGCCTCCCGCCGTTCCCAGCAGACGTCTTCATGGTGCCACAGTAT
GGATACCTCACCCTGAACAACGGGAGTCAGGCAGTAGGACGCTCT
TCATTTTACTGCCTGGAGTACTTTCCTTCTCAGATGCTGCGTACC
GGAAACAACTTTACCTTCAGCTACACTTTTGAGGACGTTCCTTTC
CACAGCAGCTACGCTCACAGCCAGAGTCTGGACCGTCTCATGAAT
CCTCTCATCGACCAGTACCTGTATttcTTGAGCAGAACAAACACT
CCAAGTGGAACCACCACGCAGTCAAGGCTTCAGTTTTCTCAGGCC
GGAGCGAGTGACATTCGGGACCAGTCTAGGAACTGGCTTCCTGGA
CCCTGTTACCGCCAGCAGCGAGTATCAAAGgtgTCTGCGGATAAC
AACAACAGTGAAttcTCGTGGACTGGAGCTACCAAGTACCACCTC
AATGGCAGAGACTCTCTGGTGAATCCGGGCCCGGCCATGGCAAGC
CACAAGGACGATGAAGAAAAGTTTTTTCCTCAGAGCGGGGTTCTC
ATCTTTGGGAAGCAAGGCTCAGAGAAAACAAATGTGGACATTGAA
AAGGTCATGATTACAGACGAAGAGGAAATCAGGACAACCAATCCC
GTGGCTACGGAGCAGTATGGTTCTGTATCTACCAACCTCCAGAGA
GGCAACctagcactcggcgaaacaacaagacctgctAGACAAGCA
GCTACCGCAGATGTCAACACACAAGGCGTTCTTCCAGGCATGGTC
TGGCAGGACAGAGATGTGTACCTTCAGGGGCCCATCTGGGCAAAG
ATTCCACACACGGACGGACATTTTCACCCCTCTCCCCTCATGGGT
GGATTCGGACTTAAACACCCTCCTCCACAGATTCTCATCAAGAAC
ACCCCGGTACCTGCGAATCCTTCGACCACCTTCAGTGCGGCAAAG
TTTGCTTCCTTCATCACACAGTACTCCACGGGACAGGTCAGCGTG
GAGATCGAGTGGGAGCTGCAGAAGGAAAACAGCAAACGCTGGAAT
CCCGAAATTCAGTACACTTCCAACTACAACAAGTCTGTTAATGTG
GACTTTACTGTGGACACTAATGGCGTGTATTCAGAGCCTCGCCCC
ATTGGCACCAGAttcCTGACTCGTAATCTGTAA

The rAAVs described herein can be used to treat optic nerve disorders and cochlear disorders, and more generally to promote nerve regeneration and survival. In one embodiment, the rAAVs described herein can be used to treat optic nerve disorders and/or retinal degenerative diseases involving retinal ganglion cell degeneration.

Hence, according to a second aspect, there is provided the recombinant vector according to the first aspect, for use as a medicament or in therapy.

According to a third aspect, there is provided the rAAV vector according to the first aspect, for use in treating, preventing or ameliorating an optic nerve disorder or a cochlear disorder, or for promoting nerve regeneration and/or survival.

In one embodiment, there is provided the rAAV vector according to the first aspect, for use in treating, preventing or ameliorating an optic nerve disorder and/or a retinal degenerative disease involving retinal ganglion cell degeneration.

According to a fourth aspect, there is provided a method of treating, preventing or ameliorating an optic nerve disorder or a cochlear disorder in a subject, or for promoting nerve regeneration and/or survival in a subject, the method comprising administering, to a subject in need of such treatment, a therapeutically effective amount of the rAAV vector according to the first aspect.

In one embodiment, there is provided a method of treating, preventing or ameliorating an optic nerve disorder and/or a retinal degenerative disease involving retinal ganglion cell degeneration, the method comprising administering, to a subject in need of such treatment, a therapeutically effective amount of the rAAV vector according to the first aspect.

In some embodiments, the rAAV vectors according to presently disclosed subject matter are used in a gene therapy technique. The BDNF encoded by the vector activates the TrkB also encoded by the vector to thereby promote survival of retinal ganglion cells (RGCs) or cochlear cells.

As illustrated in the Examples, the rAAV vectors according to the presently disclosed subject matter are able to provide a protective effect on the global retinal nerve fiber layer (RNFL) thickness composed of RGC axons, and improve their photoptic negative response (PhNR) relating to function of RGCs and their axons. Accordingly, in a preferred embodiment, the rAAV vectors according to the presently disclosed subject matter protect the global RNFL thickness composed of RGC axons. In another preferred embodiment, the rAAV vectors according to the presently disclosed subject matter improve the PhNR relating to function of RGCs and their axons (i.e. increase the PhNR amplitudes).

In one embodiment, the rAAV for use according to the third aspect, or the method according to the fourth aspect, are for preventing or treating glaucoma and glaucomatous optic neuropathy, hereditary optic neuropathy, ischemic optic neuropathy, and neurodegenerative diseases involving retinal ganglion cell degeneration. Herein, glaucoma and glaucomatous optic neuropathy comprise open angle glaucoma, normal tension glaucoma, angle-closure glaucoma, congenital glaucoma, and secondary glaucoma. Herein, hereditary optic neuropathy comprises Leber's hereditary optic neuropathy and dominantly-inherited optic atrophy. Herein, neurodegenerative diseases involving retinal ganglion cell degeneration comprise Alzheimer's disease, Parkinson's disease, Huntington's disease, and multiple system atrophy.

In some embodiments, the optic nerve disorder and/or retinal degenerative disease involving retinal ganglion cell degeneration that is treated is glaucoma. In another embodiment, the optic nerve disorder and/or retinal degenerative disease that is treated is glaucomatous optic neuropathy.

In one embodiment, the cochlear disorder which is treated may be hearing loss or deafness. The cochlear cells may be hair cells or neuronal spiral ganglion cells which send auditory signals via their axons from the ear to the brainstem. The hair cells may be inner ear hair cells or outer ear hair cells.

In another embodiment, the vectors may be used to promote nerve regeneration and/or survival.

According to a fifth aspect, there is provided a pharmaceutical composition comprising the recombinant rAAV vector according to the first aspect, and a pharmaceutically acceptable vehicle.

According to a sixth aspect, there is provided a method of preparing the pharmaceutical composition according to the fifth aspect, the method comprising contacting the recombinant rAAV vector according to the first aspect, with a pharmaceutically acceptable vehicle.

The pharmaceutical composition of the presently disclosed subject matter can be prepared by means of a method commonly used with use of a diluent commonly used in the art, that is, a diluent for agents, a carrier for agents, or the like. Examples of the dosage form of such a pharmaceutical composition comprise parenteral agents such as injections and agents for infusion. In formulation, a diluent, a carrier, an excipient, and so on according to such dosage form can be used in a pharmaceutically acceptable manner. The pharmaceutical composition according to the presently disclosed subject matter may be prepared as a sustained release formulation. In a certain embodiment, the pharmaceutical composition of the presently disclosed subject matter is administered as an injection. In a certain embodiment, the pharmaceutical composition of the presently disclosed subject matter can be administered through intraocular administration, subretinal administration, intravitreal administration, or suprachoroidal administration. In formulating the rAAV vector of the presently disclosed subject matter, a diluent, a carrier, an excipient, and so on according to such dosage form can be used in a pharmaceutically acceptable manner.

The “subject” in the prevention or treatment method of the presently disclosed subject matter is a human or non-human animal in need of such prevention or treatment, and is, in a certain embodiment, a human in need of such prevention or treatment. Examples of the “administration” to the subject comprise intraocular administration, intravitreal administration, subretinal administration, and suprachoroidal administration.

The effective amount for the rAAV vector of the presently disclosed subject matter can be appropriately optimized in view of disease severity, previous treatment, and the general health condition and age of a subject, the method of administration, other diseases, and so on. The dose of the rAAV vector of the presently disclosed subject matter can also be expressed as copy numbers of the vector genome (vg) to be administered per eye (vg/eye). vg can also be shown in genome copies (GC). In a certain embodiment, the effective dose of the rAAV vector of the presently disclosed subject matter is approximately 1×10⁶ to 1×10¹⁴ vg/eye. In one embodiment, the effective dose of the rAAV vector of the presently disclosed subject matter is approximately 1×10⁸ to 1×10¹³ vg/eye. In another embodiment, the effective dose of the rAAV vector of the presently disclosed subject matter is approximately 1×10¹⁰ to 1×10¹² vg/eye. In another embodiment, the effective dose of the rAAV vector of the presently disclosed subject matter is approximately 1×10¹¹ to 1×10¹² vg/eye.

The rAAV vector of the presently disclosed subject matter can be used in combination with a therapeutic agent or prophylactic agent for various diseases for which the therapeutic agent or prophylactic agent is expected to exhibit efficacy. In the combinational use, administrations may be carried out simultaneously, or sequentially or at desired time intervals in individual separate operations. The formulations for simultaneous administration may be a combination drug or individually formulated separate products.

The inventors have also developed a method for producing the rAAV vector according to the first aspect.

Hence, in a seventh aspect, the presently disclosed subject matter further provides a method for producing the rAAV vector according to the first aspect, the method comprising:
(i) introducing, into a rAAV vector-producing cell, a genetic construct comprising, in a 5′ to 3′ orientation:
a cytomegalovirus (CMV) promoter;
a first coding sequence, which encodes tyrosine kinase receptor B (TrkB);
a nucleotide sequence encoding a linker to generate TrkB and mBDNF as individual proteins; and
a second coding sequence, which encodes mature brain-derived neurotrophic factor (mBDNF),
   wherein the CMV promoter is operably linked to the first and second coding sequence; and
(ii) culturing the rAAV vector-producing cell, to thereby produce the rAAV vector according to the first aspect.

In some embodiments, the method for producing the rAAV comprises: introducing the genetic construct into a rAAV vector-producing cell; culturing the rAAV vector-producing cell; and collecting a culture solution from the rAAV vector-producing cell and/or a lysate of the rAAV vector-producing cell and purifying the rAAV vector from the culture solution and/or lysate.

The method for producing the rAAV vector may comprise the step of introducing the genetic construct into a rAAV vector-producing cell. The step of introducing the genetic construct into a rAAV vector-producing cell may comprise the step of introducing, in addition to the genetic construct, a plasmid comprising a Rep gene and a Cap gene and a plasmid comprising helper virus-derived genes that promote replication of AAV (e.g., adenoviral VA, E2A, E4 genes) into the rAAV vector-producing cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003). The step of introducing the genetic construct into a rAAV vector-producing cell can be carried out by using a method known to those skilled in the art.

The method for producing the rAAV vector may comprise the step of collecting a culture solution from the rAAV vector-producing cell and/or a lysate of the rAAV vector-producing cell. The lysate can be obtained, for example, by treating the rAAV vector-producing cell with a surfactant or an ultrasonic wave.

The method for producing the rAAV vector may further comprise the step of purifying the rAAV vector. To purify the rAAV vector from the lysate, for example, ion-exchange chromatography and/or hydrophobic interaction chromatography, cesium chloride density-gradient centrifugation, sucrose gradient centrifugation, iodixanol density-gradient centrifugation, ultrafiltration, diafiltration, affinity chromatography, polyethylene glycol precipitation, and ammonium sulfate precipitation may be used.

In an eighth aspect, the presently disclosed subject matter provides a rAAV vector-producing cell comprising the genetic construct of the rAAV vector of the first aspect.

Any cell that is known in the art and allows production of rAAV through introduction of a construct can be selected, without limitation, as the rAAV vector-producing cell for use in the presently disclosed subject matter. Examples of the rAAV vector-producing cell for use in the presently disclosed subject matter include various cells comprising normal cells and artificially established cells commonly used in the technical field of the presently disclosed subject matter. Examples of the rAAV vector-producing cell for use in the presently disclosed subject matter include animal cells (e.g., CHO cells, HEK293 cells, HeLa cells), insect cells (e.g., Sf9 cells), bacteria (such as Escherichia coli), and yeasts (Saccharomyces spp., Pichia spp.). In some embodiments, the rAAV vector-producing cell of the presently disclosed subject matter is an animal cell. In some embodiments, the rAAV vector-producing cell of the presently disclosed subject matter is a HEK293 cell or a cell derived therefrom (e.g., a HEK293T cell).

It will be appreciated that the presently disclosed subject matter extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No: 1-26, and so on.

Amino acid/polynucleotide/polypeptide sequences with a sequence identity which is greater than 65%, in some embodiments, greater than 70%, in some embodiments, greater than 75%, and in some embodiments, greater than 80% sequence identity to any of the sequences referred to are also envisaged. In some embodiments, the amino acid/polynucleotide/polypeptide sequence has at least 85% identity with any of the sequences referred to, in some embodiments at least 90% identity, in some embodiments at least 92% identity, in some embodiments at least 95% identity, in some embodiments at least 97% identity, in some embodiments at least 98% identity and, in some embodiments at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g., functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is one way for generating multiple alignments of proteins or DNA in accordance with the presently disclosed subject matter. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

In some embodiments, calculation of percentage identities between two amino acid/polynucleotide/polypeptide sequences may then be calculated from such an alignment as (N/T)*100, where N is the number of positions at which the sequences share an identical residue, and Tis the total number of positions compared including gaps but excluding overhangs. In some embodiments, overhangs are included in the calculation. Hence, one method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridizes to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 3 and 5.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the presently disclosed subject matter, and to show how embodiments of the same may be carried into effect, reference will now be made, by way of example, to the accompanying Figure, in which:—

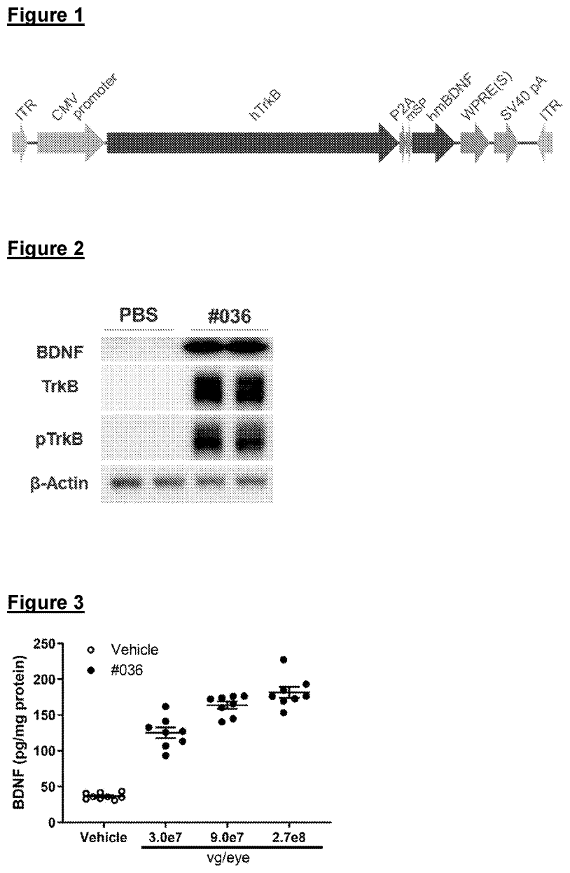

FIG. 1 shows a schematic map of the genetic construct “ITR-CMV-hTrkB-P2A-mSP-hmBDNF-WPRE(S)-SV40 pA-ITR” (SEQ ID NO: 16), which is comprised in the rAAV according to the presently disclosed subject matter, and is referred to throughout the Examples as ‘#036’.

FIG. 2 shows results of Western blot analysis for expression of transgene products (hmBDNF, TrkB) and the presence of activated TrkB (phospho-TrkB: pTrkB) in HEK293 cells 2 days after transduction with rAAV #036 shown in FIG. 1 (n=2). In the figure, rAAV #036 is expressed as “#036”, and hmBDNF is expressed as “BDNF”.

FIG. 3 shows results of ELISA for expression levels of a transgene product (hmBDNF) in mouse retinal tissues 3 weeks after intravitreal administration of rAAV #036 shown in FIG. 1 at a dose of 3.0×10⁷ (3.007) vg/1 μL, 9.0×10⁷ (9.0e7) vg/1 μL, or 2.7×10⁸ (2.7e8) vg/1 μL per eye. Bars in the graph represent mean±standard error of the mean for each group (n=8 or 9). In the figure, rAAV #036 is expressed as “#036”, and hmBDNF is expressed as “BDNF”.

FIG. 4 shows results of Western blot analysis for expression of transgene products (hmBDNF, TrkB) and the presence of activated TrkB (pTrkB) in mouse retinal tissues 3 weeks after intravitreal administration of rAAV #036 shown in FIG. 1 at a dose of 2.7×10⁸ (2.7e8) vg/1 μL per eye (n=3). In the figure, rAAV #036 is expressed as “#036”, and hmBDNF is expressed as “BDNF”.

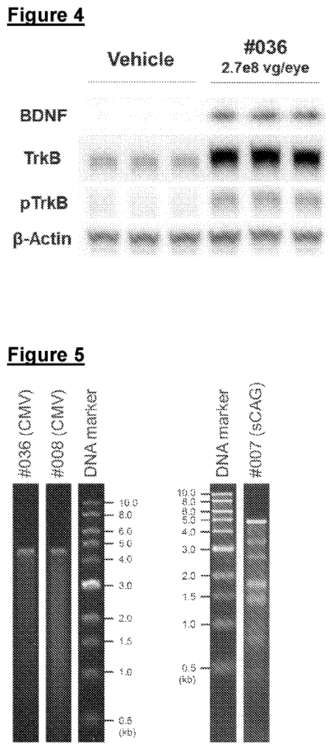

FIG. 5 shows results of alkaline agarose gel electrophoresis analysis for genomic DNA of rAAV #007 (sCAG-hTrkB-P2A-SP-hmBDNF-WPRE(S)-SV40 pA), rAAV #008 (CMV-hTrkB-P2A-SP-hmBDNF-WPRE(S)-SV40 pA), and rAAV #036 (CMV-hTrkB-P2A-mSP-hmBDNF-WPRE(S)-SV40 pA). The overall genome lengths of rAAV #007, rAAV #008, and rAAV #036 are approximately 4.8 kb, approximately 4.6 kb, and approximately 4.6 kb, respectively.

FIG. 6 shows productivity of rAAV #007, rAAV #008, and rAAV #036. The vertical axis shows relative titer of vector genome concentrations of rAAV #008 compared to rAAV #007, and rAAV #036 compared to rAAV #008 (calculated with ITR primers) in cell lysates.

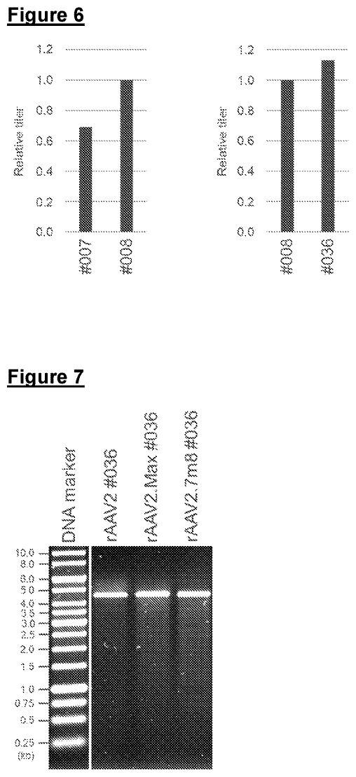

FIG. 7 shows results of alkaline agarose gel electrophoresis analysis for genomic DNA of rAAV #036, rAAV2.Max #036, and rAAV2.7m8 #036. The overall genome lengths of rAAV #036, rAAV2.Max #036, and rAAV2.7m8 #036 are approximately 4.6 kb.

FIG. 8 shows productivity of rAAV #036, rAAV2.Max #036, and rAAV2.7m8 #036. The vertical axis shows relative titer of vector genome concentrations of rAAV #036, rAAV2.Max #036, and rAAV2.7m8 #036 (calculated with ITR primers) in cell lysates.

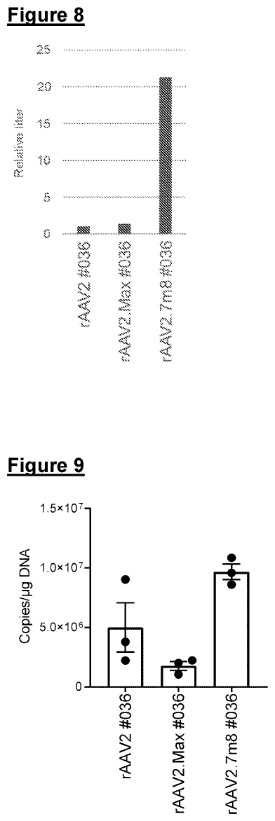

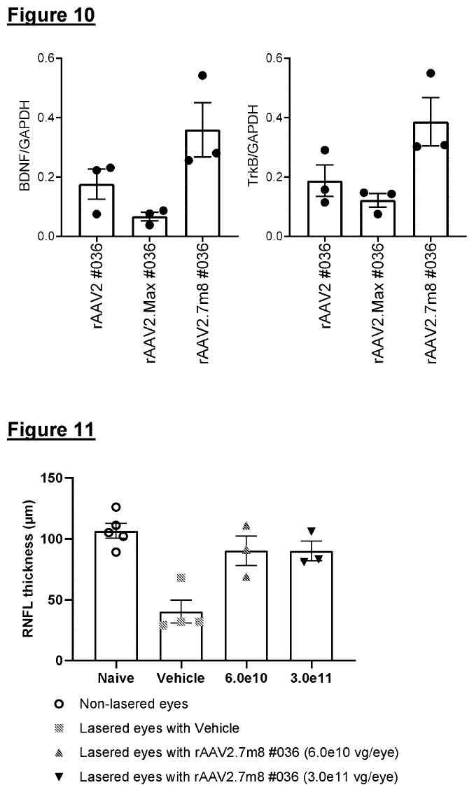

FIG. 9 shows vector copy number (copies/μg DNA) using real-time PCR in monkey retinal tissues 8 weeks after intravitreal administration of rAAV #036, rAAV2.Max #036, and rAAV2.7m8 #036, at a dose of 6.3×10¹⁰ vg/70 μL per eye. Bars in the graph represent mean±standard error of the mean for each group (n=3).

FIG. 10 shows RNA expression levels of BDNF and TrkB corrected with GAPDH using real-time PCR in monkey retinal tissues 8 weeks after intravitreal administration of rAAV #036, rAAV2.Max #036, and rAAV2.7m8 #036, at a dose of 6.3×10¹⁰ vg/70 μL per eye. Bars in the graph represent mean±standard error of the mean for each group (n=3).

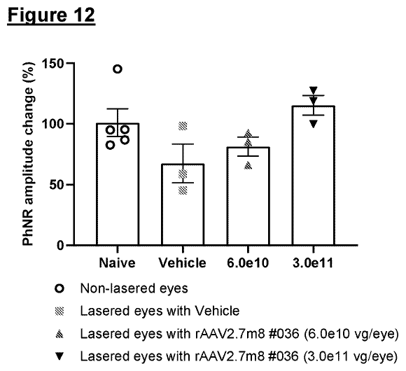

FIG. 11 shows global retinal nerve fiber layer (RNFL) thicknesses using optical coherence tomography (OCT) circular scanning of optic nerve heads in non-laser-treated eyes and laser-treated eyes after intravitreal administration of vehicle or rAAV2.7m8 #036 at a dose of 6.0×10¹⁰ (6.0e10) vg/70 μL or 3.0×10¹¹ (3.0e11) vg/70 μL per eye. Bars in the graph represent mean±standard error of the mean for each group (n=3 to 5).

FIG. 12 shows percentage change of photopic negative response (PhNR) amplitude from the pre-administration using focal electroretinogram on the fovea in non-laser-treated eyes and laser-treated eyes after intravitreal administration of vehicle or rAAV2.7m8 #036 at a dose of 6.0×10¹⁰ (6.0e10) vg/70 μL or 3.0×10¹¹ (3.0e11) vg/70 μL per eye. Bars in the graph represent mean±standard error of the mean for each group (n=3 or 5).

### EXAMPLES

The present inventors observed an important discrepancy in the yield when manufacturing some of the rAAV vectors described in WO 2017/072498 and Hum. Gene Ther., 2018. 29(7): p. 828-841. In particular, the inventors observed a significant problem in which rAAV vectors comprising a TrkB gene and a BDNF gene, as designed in accordance with the teaching of the prior art documents, showed fragmentation or truncation of rAAV genomic DNA in the production process. The occurrence of the truncation of genomic DNA interferes with efficient production of a rAAV vector comprising a TrkB gene and a BDNF gene, resulting in lowered production efficiency of the rAAV vector. As such, the inventors set out to set out to obtain a rAAV vector comprising both a TrkB gene and a BDNF gene, with reduced truncation of genomic DNA.

### Example 1—Production of rAAV Construct

A plasmid including a truncated CAG (short CAG: sCAG) promoter (0.8 kb) was designed according to the descriptions of International Publication No. WO 2017/072498 and Hum. Gene Ther., 2018. 29 (7): p. 828-841, and pAAV-sCAG-hTrkB-P2A-SP-hmBDNF-WPRE(S)-SV40 pA (SEQ ID No: 24) was obtained (this plasmid construct is also referred to as #007). Plamid construct pAAV-CMV-hTrkB-P2A-SP-hmBDNF-WPRE(S)-SV40 pA (SEQ ID No: 25), which includes CMV promoter (SEQ ID No: 1), was obtained (this plasmid construct is also referred to as #008). Plasmid construct pAAV-CMV-hTrkB-P2A-mSP-hmBDNF-WPRE(S)-SV40PA (SEQ ID No: 26, in which the signal peptide is modified from #008) was obtained (this plasmid construct is also referred to as #036).

Plasmid construct #036 contains the polynucleotide “ITR-CMV-hTrkB-P2A-mSP-hmBDNF-WPRE(S)-SV40 pA-ITR” (SEQ ID NO: 16), which comprises the polynucleotide “CMV-hTrkB-P2A-mSP-hmBDNF-WPRE(S)-SV40 pA” (SEQ ID No: 15). The polynucleotide “CMV-hTrkB-P2A-mSP-hmBDNF” (SEQ ID No: 14) is a region spanning from the CMV promoter to the nucleotide sequence encoding hmBDNF in SEQ ID NO: 15. In addition, FIG. 1 shows the map of the polynucleotide “ITR-CMV-hTrkB-P2A-mSP-hmBDNF-WPRE(S)-SV40 pA-ITR” (SEQ ID No: 16) included in the plasmid construct #036.

rAAV2 vectors were produced with the plasmid construct #007 (including an sCAG promoter), the plasmid construct #008 (including a CMV promoter), and the plasmid construct #036. The rAAV2s produced are referred to as rAAV #007, rAAV #008, and rAAV #036, respectively. rAAV2.7m8 was produced with the plasmid construct #036 and referred to as rAAV2.7m8 #036. rAAV2 Max was produced with the plasmid construct #036 and referred to as rAAV2 Max #036. rAAV2.7m8 has a capsid which comprises the amino acid sequence of SEQ ID No: 18. rAAV2 Max has a capsid which comprises the amino acid sequence of SEQ ID No: 19.

### Example 2-Expression of Transgene Products and Activation of TrkB in HEK293 Cells Transduced with rAAV #036

HEK293 cells were seeded on a collagen I coated 24-well microplate (Iwaki, catalog No. 4820-010) at 1×10⁵ cells/well 1 day before the rAAV transduction experiment, and subjected to static culture in Dulbecco's Modified Eagle Medium (DMEM, Sigma-Aldrich Co. LLC, catalog No. D6429) containing 10% fetal bovine serum (FBS, Hyclone, catalog No. SH30070.03) and 1% penicillin-streptomycin (Thermo Fisher Scientific, catalog No. 15070-063) under conditions of 37° C. and 5% CO₂.

One day after the cell seeding, the whole medium was replaced with 425 μL of DMEM containing 1% FBS and 1% penicillin-streptomycin, and 75 μL of rAAV #036 or Dulbecco's Phosphate Buffered Saline (DPBS, Wako Pure Chemical Industries, Ltd., catalog No. 045-29795) was added dropwise to the cells, which was subjected to static culture under conditions of 37° C. and 5% CO₂. For the dropwise addition, rAAV #036 had been prepared in advance to reach a final concentration of 2.5×10⁹ vg/mL with DPBS.

Two days after the addition of rAAV, the cells were washed with DPBS, a cell lysis buffer was then added thereto, and the lysate was collected and stored at −80° C. The cell lysis buffer had been prepared to reach final concentrations of 20 mM N-2-hydroxyethylpiperazine-N′-2-ethane sulfonic acid (HEPES, Thermo Fisher Scientific, catalog No. 15630-080), 135 mM sodium chloride (NaCl, Wako Pure Chemical Industries, Ltd., catalog No. 191-01665), 1% Triton® X-100 (Nacalai Tesque, Inc., catalog No. 35501-15), 0.1% Benzonase® Nuclease (Merck Millipore, catalog No. 70664), and 1% Halt™ Protease and Phosphatase Inhibitor Cocktail (Thermo Fisher Scientific, catalog No. 78441).

Thawed lysate was left to stand on ice for 20 minutes and then centrifuged by using a centrifuge (Hitachi, Ltd.) at 4° C. and 15000 rpm for 5 minutes, and the supernatant was used for the subsequent tests. Protein concentrations of the samples were measured with Pierce™ BCA Protein Assay Kit (Thermo Fisher Scientific, catalog No. 23227) and were determined from absorbance at 562 nm with a microplate reader (SpectraMax Plus 384, Molecular Devices, LLC.).

Western blot, using equal amounts of protein among samples, was performed to confirm expressions of transgene products (hmBDNF and TrkB) and activation of TrkB (phosphorylated TrkB (phospho TrkB: pTrkB)) in HEK293 cells. The following antibodies were used for the detection; primary antibodies used were rabbit anti-BDNF [EPR1292] antibody (Abcam plc., catalog No. ab108319), rabbit anti-TrkB [80E3] antibody (Cell Signaling Technology: CST, catalog No. 4603S), rabbit anti-phospho-TrkB [Tyr515] polyclonal antibody (Thermo Fisher Scientific, catalog No. PA5-36695), and rabbit anti-β-Actin antibody (Cell Signaling Technology, catalog No. 4967S); secondary antibodies used were ECL™ anti-rabbit IgG and HRP-Linked F(ab′)2 fragment (from donkey) (GE Healthcare, catalog No. NA934V). Amersham™ ECL™ Prime Western Blotting Detection Reagents (GE Healthcare, catalog No. RPN2232) were used for the detection in Western blot, and images were acquired by using a ChemiDoc Touch imaging system (Bio-Rad Laboratories, Inc.). Expressions of hmBDNF and TrkB as transgene products and activation of TrkB were confirmed in the cells transduced with rAAV #036 (FIG. 2). In the figure, rAAV #036 is expressed as “#036”, and hmBDNF is expressed as “BDNF”.

### Example 3: Expression of Transgene Products and Activation of TrkB in Mouse Retinal Tissue Intravitreally Administered with rAAV #036

A vehicle or rAAV #036 was intravitreally administered to 5-week-old male C57BL/6J mice (Charles River Laboratories Japan, Inc.), and expression levels of BDNF in the retinal tissues 3 weeks after the administration were analysed. A solution obtained by adding 0.001% Pluronic™ F-68 (Thermo Fisher Scientific, catalog No. 24040032) to DPBS was used as a vehicle. rAAV #036 was intravitreally administered at a dose of 3.0×10⁷ (3.0e7) vg/1 μL, 9.0×10⁷ (9.0e7) vg/1 μL, or 2.7×10⁸ (2.7e8) vg/1 μL per eye. A glass pipette (Sankyo Medic Co., Ltd.) connected to the microinjector FemtoJet® 4i (Eppendorf) was inserted under anesthesia into the vitreous body of each 5-week-old C57BL/6J mouse, and 1 μL was administered per eye. Three weeks after the administration, each mouse was euthanized by bleeding under anesthesia with isoflurane, and the retinal tissue was sampled. After the retinal tissue sampled was frozen with dry ice, the same cell lysis buffer as used for the analysis of transgene products expression in cultured cells in Example 2 was added thereto, and the resultant was then homogenized by using BioMasher (Nippi, Incorporated, catalog No. 320103) and stored at −80° C.

Thawed lysate was left to stand on ice for 20 to 30 minutes and then centrifuged by using a centrifuge (Hitachi, Ltd.) at 4° C. and 15000 rpm for 10 minutes, and the supernatant was used for the subsequent tests. Protein concentrations of the samples were measured with Pierce™ BCA Protein Assay Kit and were determined from absorbance at 562 nm with a microplate reader. Protein expression level of hmBDNF was determined by calculating the amount of hmBDNF protein using Human Free BDNF Quantikine® ELISA Kit (R&D Systems, Inc., catalog No. DBDoo) from absorbance with a microplate reader (a value of absorbance at 450 nm minus absorbance at 540 nm was employed) and then corrected with the total protein concentration (FIG. 3).

As demonstrated in FIG. 3, expression of hmBDNF was confirmed in mouse retinal tissues upon intravitreal administration of rAAV #036. Further, expression of transgene products (hmBDNF and TrkB) and activation of TrkB in retinae upon administration of rAAV #036 were evaluated by using Western blot. For this evaluation, high-dose (2.7×10⁸ vg/1 μL) rAAV #036 administration and vehicle administration groups were subjected, and three samples in each group that show closest value to the median value in hmBDNF expression analysis using ELISA were selected. Reagents and procedures used in this evaluation were identical to those in Example 2. Expression of hmBDNF and TrkB as transgene products and activation of TrkB (pTrkB) were confirmed in the mouse retinal tissues transduced with rAAV #036 (FIG. 4). In the figure, rAAV #036 is expressed as “#036”, and hmBDNF is expressed as “BDNF”.

### Example 4: RAAV Genomic DNA Analysis

After the subsequent treatment with DNase I and then with Proteinase K, the AAV genomic DNA of rAAV #036 was purified by isopropanol precipitation. The DNA concentration was measured using a fluorometer (Thermo Fisher Scientific, Qubit® Fluorometer and Qubit® dsDNA HS Assay Kit), and 160 ng of the AAV genomic DNA was analysed by electrophoresis on an alkaline agarose gel containing 50 mM sodium hydroxide (NaOH). The AAV genomic DNA and DNA size markers used for the electrophoresis had been denatured in the presence of 50 mM NaOH/0.3% SDS at 95° C. for 5 to 10 minutes.

The gel after the electrophoresis was stained with a reagent for staining single-stranded DNA (Biotium, catalog No. 41003, GelRed™), and the DNA was detected with a UV transilluminator (Bio-Rad Laboratories, Inc., ChemiDoc MP Imaging System) (FIG. 5). AAV genomic DNA analysis was conducted also for rAAV #007 and rAAV #008 in the same manner, except that, for rAAV #007, purification of genomic DNA was carried out by using a DNA purification column (QIAGEN, catalog No. 28104, QIAquick® PCR Purification Kit). In electrophoresis, 200 ng of genomic DNA was used for rAAV #007 and rAAV #008. As demonstrated in FIG. 5, it was confirmed that the truncation of genomic DNA in rAAV #036 and that in rAAV #008 (both including a CMV promoter) were reduced compared with that found for rAAV vectors including an sCAG promoter designed according to the descriptions of International Publication No. WO 2017/072498 and Hum. Gene Ther., 2018. 29 (7): p. 828-841 (e.g., rAAV #007).

### Example 5: Evaluation of rAAV Productivity

0.2% Triton X-100 and 200 mM NaCl (both at their final concentrations) were added into the culture solutions of production cells for rAAV #007 and rAAV #008 to obtain cell lysates. After the subsequent treatment with DNase I and Exo I, protease treatment and purification of AAV genomic DNA were carried out by using a QIAamp MinElute Virus Spin Kit (QIAGEN, catalog No. 57704). Next, real-time PCR was carried out using an AAVpro® Titration Kit for Real Time PCR (Takara Bio Inc., catalog No. 6233) and ITR primers attached to the kit. A calibration curve was prepared by using standard DNA attached to the kit, and relative titer of vector genome concentration in the cell lysates were calculated (FIG. 6).

For rAAV #008 and rAAV #036, cell lysates 5 days after the transfection were obtained, and vg contained in each cell lysate was quantified in the same manner, except that after DNase I/Exo I treatment, AAV genomic DNA was extracted by Proteinase K treatment, the solution was diluted with water, and real-time PCR was then performed.

As demonstrated in FIG. 6, it was confirmed that enhanced productivity is achieved with rAAV #008, which included a CMV promoter, as compared with that with rAAV vectors comprising a sCAG promoter designed according to the descriptions of International Publication No. WO 2017/072498 and Hum. Gene Ther., 2018. 29 (7): p. 828-841 (e.g., rAAV #007). In addition, rAAV #036 was confirmed to exhibit high productivity as rAAV #008.

### Example 6: RAAV2.7m8 Vector and rAAV2 Max Vector

AAV genomic DNA analysis and evaluation of rAAV productivity were conducted for rAAV2.Max #036 and rAAV2.7m8 #036. The genome integrity of rAAV2.Max #036 and rAAV2.7m8 #036 was confirmed (FIG. 7). The relative titer contained in each cell lysate was quantified. It was confirmed that enhanced productivity is achieved with rAAV2.7m8 #036 compared with rAAV #036 and rAAV2.Max #036 (FIG. 8).

### Example 7: Expression of Transgene Products in Monkey Retinal Tissue Intravitreally Administered with rAAV2.7m8 Vector and rAAV2 Max Vector

rAAV #036, rAAV2.Max #036, and rAAV2.7m8 #036 were intravitreally administered to female cynomolgus monkeys (Shin Nippon Biomedical Laboratories, Ltd) at a dose of 6.3×10¹⁰ vg/70 μL per eye. A 30G MYSHOT™ Insulin Syringe (NIPRO Pharma Vietnam Co., Ltd.) was inserted under anesthesia into the vitreous body of each monkey, and 70 μL was administered per eye. Eight weeks after the administration, each monkey was euthanized by bleeding under anesthesia, and the retinal tissue was sampled. After the retinal tissue samples were frozen, DNA and RNA were isolated using NucleoSpin® RNA/Protein (Takara Bio Inc., catalog No. 740933) and NucleoSpin® RNA/DNA Buffer Set (Takara Bio Inc., catalog No. 740944) after homogenization using BioMasher.

DNA and RNA concentrations of the samples were measured with NanoDrop™ 8000 Spectrophotometer (Thermo Fisher Scientific). The vector copy number and RNA expression levels were analyzed by real-time PCR with Power SYBR™ Green PCR Master Mix (Thermo Fisher Scientific, catalog No. 4368708). The vector copy number was calculated using a primer which was designed on the sequence of the CMV promoter in SEQ ID No: 26. The primers of RNA were designed on the sequences of BDNF and TrkB in SEQ ID No: 26, respectively. RNA expression levels of BDNF and TrkB were normalized by GAPDH.

Enhanced vector copy number was observed with rAAV2.7m8 #036 compared with rAAV #036 and rAAV2.Max #036 in monkey retina (FIG. 9). Similarly, enhanced RNA expression levels of BDNF and TrkB were observed with rAAV2.7m8 #036 compared with rAAV #036 and rAAV2.Max #036 in monkey retina (FIG. 10). Accordingly, these data show that the rAAV vector according to the presently disclosed subject matter demonstrates increased transduction efficiency, and can increase RNA expression levels of BDNF and TrkB in retinal tissues when administered in vivo.

### Example 8: Efficacy of rAAV2.7m8 #036 on Retinal Ganglion Cell (RGC) Related Structure and Function in a Monkey Ocular Hypertension Model

In four to nine years old male cynomolgus monkeys (Shin Nippon Biomedical Laboratories, Ltd) were used as an experimental glaucoma model, with a laser being applied at a wavelength of 532 nm for uniform 360-degree irradiation around the trabecular meshwork, as previously described (Ophthalmic Res., 2017. 58(2): 99-106). Laser-treated eyes with elevated intraocular pressure (IOP) compared with non-laser-treated eyes was confirmed, as seen in the previous report. Vehicle or rAAV2.7m8 #036 at a dose of 6.0×10¹⁰ (6.0e10) vg/70 μL or 3.0×10¹¹ (3.0e11) vg/70 μL per eye was intravitreally administered following 19 days from the laser application. A solution obtained by adding 0.01% Poloxamer188 (Merck Millipore, catalog No. 137097) to PBS was used as a vehicle. A 30G MYSHOT™ Insulin Syringe or BD Insulin Syringes with BD Ultra-Fine™ 8 mm×30G needle (Becton Dickinson & Co.) was inserted under anesthesia into the vitreous body of each monkey, and 70 μL was administered per eye. Retinal nerve fiber layer (RNFL) thickness around the optic nerve head and photopic negative response (PhNR) were measured in bilateral eyes in each monkey under anesthesia 16 weeks after laser application. The bilateral optic nerve heads were circularly scanned and global RNFL thicknesses were measured with a Spectralis® optical coherence tomography (OCT) device (Heidelberg Engineering Ltd.) as previously described (Ophthalmic Res., 2017. 58(2): 99-106). Focal electroretinogram on the fovea was measured by photic stimulation (duration: 100 ms, stimulate light: 5, background light: 5, stimulate light size: 15°, intensity: 3.082 cds/m², background light: white) using Kowa ER-80 (Kowa Co., Ltd.) and PuREC (PC100-A, Mayo Ltd.) after placing a contact lens-type electrode on the cornea. PhNR is a slow negative-going wave to reflect the activity of RGCs and their axons, and reduced PhNR amplitudes have been reported in patients with glaucoma (Doc Ophthalmol., 2018. 136(3): 207-211; Invest Ophthalmol Vis Sci., 2008. 49:2201-2207). PhNR amplitudes were measured from the peak of the b-wave to the maximum amplitude in trough immediately after i-wave as described (Doc Ophthalmol., 2018. 136(3): 207-211).

The protective effect of rAAV2.7m8 #036 on global RNFL thickness in the laser-treated eyes was observed, with the global RNFL thickness remaining similar to those of the non-lasered eyes. In contrast, the global RNFL thickness in the laser-treated eyes administered with the vehicle was reduced compared with the non-laser-treated eyes (FIG. 11). The protective effect of rAAV2.7m8 #036 on the percentage change of PhNR amplitude from the pre-administration in the laser-treated eyes was also observed. In contrast, the PhNR percentage change in the laser-treated eyes with the vehicle was reduced compared with the non-laser-treated eyes (FIG. 12). Accordingly, these data show that the rAAV vector according to the presently disclosed subject matter demonstrates a protective effect on RGC related structure and function in experimental monkey models of glaucoma.

### Conclusions

As demonstrated throughout the Examples, the inventors discovered that a rAAV vector carrying a cytomegalovirus (CMV) promoter operably linked to a naturally occurring TrkB gene and a naturally occurring mature BDNF gene, demonstrated reduced fragmentation/truncation of genomic DNA. It means that the rAAV vector of the claimed presently disclosed subject matter, comprising a CMV promoter operably linked to naturally occurring TrkB and mBDNF, can be produced with increased production efficiency and yields, increased transduction efficiency to retina, and demonstrates a protective effect on RGC related structure and function in experimental monkey models of glaucoma.

### REFERENCES

1. International Publication No. WO 2017/072498
2. International Publication No. WO 2018/185468
3. Hum. Gene Ther., 2018. 29(7): p. 828-841
4. Cell Death Dis., 2018. 9:1007

1. A recombinant adeno-associated virus (rAAV) vector comprising a genetic construct comprising, in a 5′ to 3′ orientation:
a cytomegalovirus (CMV) promoter;
a first coding sequence, which encodes tyrosine kinase receptor B (TrkB);
a nucleotide sequence encoding a linker to generate TrkB and mature brain-derived neurotrophic factor (mBDNF) as individual proteins; and
a second coding sequence, which encodes mBDNF,
wherein the CMV promoter is operably linked to the first and second coding sequences.

2. The rAAV vector according to claim 1, wherein the CMV promoter comprises a nucleotide sequence as set out in SEQ ID No: 1, or a fragment or variant thereof.

3. The rAAV vector according to claim 1, wherein the first coding sequence encodes naturally occurring TrkB, or a variant having the function thereof.

4. The rAAV vector according to claim 1, wherein the first coding sequence encodes an amino acid sequence as set out in SEQ ID No: 2, or a fragment or variant thereof, and/or wherein the first coding sequence comprises a nucleotide sequence as set out in SEQ ID No: 3, or a fragment or variant thereof.

5. The rAAV vector according to claim 1, wherein the second coding sequence encodes naturally occurring mBDNF.

6. The rAAV vector according to claim 1, wherein the second coding sequence encodes an amino acid sequence as set out in SEQ ID No: 4, or a fragment or variant thereof, and/or wherein the second coding sequence comprises a nucleotide sequence as set out in SEQ ID No: 5, or a fragment or variant thereof.

7. The rAAV vector according to claim 1, wherein the genetic construct further comprises a nucleotide sequence encoding a signal peptide, optionally wherein the signal peptide is positioned on the 5′ side of the nucleotide sequence encoding mBDNF, and/or wherein the the nucleotide sequence encoding the signal peptide is positioned on the 3′ side of the nucleotide sequence encoding the linker.

8. The rAAV vector according to claim 7, wherein the nucleotide sequence encoding the signal peptide encodes an amino acid sequence as set out in SEQ ID No: 6 or SEQ ID No: 20, or a fragment or variant thereof, and/or wherein the signal peptide comprises a nucleotide sequence as set out in SEQ ID No: 7 or SEQ ID No: 21, or a fragment or variant thereof.

9. The rAAV vector according to claim 1, wherein the linker is a P2A peptide.

10. The rAAV vector according to claim 1, wherein the nucleotide sequence encoding the linker encodes an amino acid sequence as set out in SEQ ID No: 8, or a fragment or variant thereof, and/or wherein the linker comprises a nucleotide sequence as set out in SEQ ID No: 9, or a fragment or variant thereof.

11. The rAAV vector according to claim 1, wherein the genetic construct further comprises a nucleotide sequence encoding a woodchuck hepatitis virus post-transcriptional regulatory element (WPRE), optionally wherein the WPRE comprises a nucleotide sequence as set out in SEQ ID No: 10, or a fragment or variant thereof.

12. The rAAV vector according to claim 1, wherein the genetic construct further comprises a nucleotide sequence encoding a polyA signal sequence, optionally wherein the polyA signal sequence comprises a nucleotide sequence as set out in SEQ ID No: 11, or a fragment or variant thereof.

13. The rAAV vector according to claim 1, wherein the rAAV vector comprises a genetic construct comprising, in a 5′ to 3′ direction, a CMV promoter sequence, a first coding sequence encoding TrkB, a nucleotide sequence encoding a P2A linker peptide, a nucleotide sequence encoding a signal peptide, a second coding sequence encoding mBDNF, a woodchuck hepatitis virus post-transcriptional regulatory element (WPRE), and a simian virus 40 (SV40) polyA signal sequence.

14. The rAAV vector according to claim 1, wherein the genetic construct comprises a nucleotide sequence as set out in any one of SEQ ID No: 14 to 17, or a variant or fragment thereof.

15. The rAAV vector according to claim 1, wherein the rAAV vector is a rAAV2 vector.

16. The rAAV vector according to claim 1, wherein the rAAV vector is a rAAV2.7m8 vector.

17. The rAAV vector according to claim 1, for use as a medicament or in therapy.

18. The rAAV vector according to claim 1, for use in treating, preventing or ameliorating an optic nerve disorder and/or a retinal degenerative disease involving retinal ganglion cell degeneration.

19. The rAAV vector according to claim 18, wherein the optic nerve disorder and/or retinal degenerative disease involving retinal ganglion cell degeneration is glaucoma or glaucoma optic neuropathy.

20. A method of treating, preventing or ameliorating an optic nerve disorder and/or a retinal degenerative disease involving retinal ganglion cell degeneration in a subject, the method comprising administering, to a subject in need of such treatment, a therapeutically effective amount of the rAAV vector according to claim 1.

21. A pharmaceutical composition comprising the recombinant rAAV vector according to claim 1, and a pharmaceutically acceptable vehicle.

22. A method of preparing the pharmaceutical composition according to claim 21, the method comprising contacting the recombinant rAAV vector according to claim 1, with a pharmaceutically acceptable vehicle.

23. A method for producing the rAAV vector according to claim 1, the method comprising:
(i) introducing, into a rAAV vector-producing cell, a genetic construct comprising, in a 5′ to 3′ orientation:
a cytomegalovirus (CMV) promoter;
a first coding sequence, which encodes tyrosine kinase receptor B (TrkB);
a nucleotide sequence encoding a linker to generate TrkB and mature brain-derived neurotrophic factor (mBDNF) as individual proteins; and
a second coding sequence, which encodes mBDNF,
wherein the CMV promoter is operably linked to the first and second coding sequence; and
(ii) culturing the rAAV vector-producing cell, to thereby produce the rAAV vector according to claim 1.

24. A rAAV vector-producing cell comprising the genetic construct of the rAAV vector of claim 1.


SEQUENCE LISTING (26 sequences)

SEQ ID NO: 1 - DNA, 571 bp, synthetic construct
  source 1..571: mol_type = other DNA; note = CMV promoter
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtg

SEQ ID NO: 2 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 3 - DNA, 2466 bp, Homo sapiens
  source 1..2466: mol_type = genomic DNA
atgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggc

SEQ ID NO: 4 - protein, 119 aa, Homo sapiens
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 5 - DNA, 357 bp, Homo sapiens
  source 1..357: mol_type = genomic DNA
cactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 6 - protein, 18 aa, synthetic construct
  source 1..18: note = nv3 signal peptide
MRILLLTMVISYFGCMKA

SEQ ID NO: 7 - DNA, 54 bp, synthetic construct
  source 1..54: mol_type = other DNA; note = nv3 signal peptide
atgcggatccttctgcttactatggttatttcatactttggttgcatgaaggct

SEQ ID NO: 8 - protein, 21 aa, synthetic construct
  source 1..21: note = SG-P2A peptide
SGATNFSLLKQAGDVEENPGP

SEQ ID NO: 9 - DNA, 63 bp, synthetic construct
  source 1..63: mol_type = other DNA; note = SG-P2A peptide
agcggcgccacaaatttctccctgctgaagcaggcaggcgacgtggaggagaaccctggacca

SEQ ID NO: 10 - DNA, 247 bp, synthetic construct
  source 1..247: mol_type = other DNA; note = WPRE(S)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 11 - DNA, 212 bp, Simian virus 40
  source 1..212: mol_type = other DNA
agacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctaca

SEQ ID NO: 12 - DNA, 130 bp, synthetic construct
  source 1..130: mol_type = other DNA; note = 5'ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 13 - DNA, 130 bp, synthetic construct
  source 1..130: mol_type = other DNA; note = 3'ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 3527 bp, synthetic construct
  source 1..3527: mol_type = other DNA; note = CMV-hTrkB-P2A-mSP-hmBDNF
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgcggatccttctgcttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 15 - DNA, 4069 bp, synthetic construct
  source 1..4069: mol_type = other DNA; note = CMV-hTrkB-P2A-mSP-hmBDNF-WPRE(S)-SV40pA
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgcggatccttctgcttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatg

SEQ ID NO: 16 - DNA, 4557 bp, synthetic construct
  source 1..4557: mol_type = other DNA; note = ITR-CMV-hTrkB-P2A-mSP-hmBDNF-WPRE(S)-SV40pA-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttgtagttaatgattaacccgccatgctacttatctacgtagccatgctctagtatcgatatcaagctttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgcggatccttctgcttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcatgaggttttttaaagcaagtaaaacctctacaaatgtggtaaaatccgataaggactagagcatggctacgtagataagtagcatggcgggttaatcattaactacaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 4489 bp, synthetic construct
  source 1..4489: mol_type = other DNA; note = ITR-CMV-hTrkB-P2A-mSP-hmBDNF-WPRE(S)-SV40pA-ITR (2)
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgcggatccttctgcttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcatgaggttttttaaagcaagtaaaacctctacaaatgtggtaaaatccgataaggactagagcatggctacgtagataagtagcatggcgggttaatcattaactacaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - protein, 745 aa, synthetic construct
  source 1..745: note = rAAV2.7m8 vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 745 aa, synthetic construct
  source 1..745: note = rAAV2 Max vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 20 - protein, 18 aa, synthetic construct
  source 1..18: note = BDNF signal peptide
MTILFLTMVISYFGCMKA

SEQ ID NO: 21 - DNA, 54 bp, synthetic construct
  source 1..54: mol_type = other DNA; note = BDNF signal peptide
atgaccatccttttccttactatggttatttcatactttggttgcatgaaggct

SEQ ID NO: 22 - DNA, 2238 bp, synthetic construct
  source 1..2238: mol_type = other DNA; note = rAAV2.7m8 vector
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaaacaacaagacctgctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 23 - DNA, 2238 bp, synthetic construct
  source 1..2238: mol_type = other DNA; note = rAAV2 Max vector
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcacttctttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttgagcagaacaaacactccaagtggaaccaccacgcagtcaaggcttcagttttctcaggccggagcgagtgacattcgggaccagtctaggaactggcttcctggaccctgttaccgccagcagcgagtatcaaaggtgtctgcggataacaacaacagtgaattctcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaaacaacaagacctgctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagattcctgactcgtaatctgtaa

SEQ ID NO: 24 - DNA, 7882 bp, synthetic construct
  source 1..7882: mol_type = other DNA; note = pAAV-sCAG-hTrkB-P2A-SP-hmBDNF-WPRE(S)-SV40pA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttgtagttaatgattaacccgccatgctacttatctacgtagccatgctctagacatggctcgacagatcgagctccaccgggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgctgctagcttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgaccatccttttccttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcatgaggttttttaaagcaagtaaaacctctacaaatgtggtaaaatccgataaggactagagcatggctacgtagataagtagcatggcgggttaatcattaactacaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 25 - DNA, 7651 bp, synthetic construct
  source 1..7651: mol_type = other DNA; note = pAAV-CMV-hTrkB-P2A-SP-hmBDNF-WPRE(S)-SV40pA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttgtagttaatgattaacccgccatgctacttatctacgtagccatgctctagtatcgatatcaagctttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgaccatccttttccttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcatgaggttttttaaagcaagtaaaacctctacaaatgtggtaaaatccgataaggactagagcatggctacgtagataagtagcatggcgggttaatcattaactacaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 26 - DNA, 7651 bp, synthetic construct
  source 1..7651: mol_type = other DNA; note = pAAV-CMV-hTrkB-P2A-mSP-hmBDNF-WPRE(S)-SV40pA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagctttgtagttaatgattaacccgccatgctacttatctacgtagccatgctctagtatcgatatcaagctttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtggatatccttaagcatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcagcggcgccacaaatttctccctgctgaagcaggcaggcgacgtggaggagaaccctggaccaatgcggatccttctgcttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcatgaggttttttaaagcaagtaaaacctctacaaatgtggtaaaatccgataaggactagagcatggctacgtagataagtagcatggcgggttaatcattaactacaagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctcgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag